(19) United States
(12) Patent Application Publication
(10) Pub. No.: US 2026/0345648 A1
(43) Pub. Date: Nov. 5, 2026

(54) DOSING STRATEGY TO MAXIMIZE MUCOSAL TISSUE UPTAKE OF PROPHYLACTIC INTRARECTAL TENOFOVIR

(71) Applicant: THE JOHNS HOPKINS UNIVERSITY, Baltimore, MD (US)

(72) Inventors: Craig W. HENDRIX, Ellicott City, MD (US); Edward FUCHS, Westminster, MD (US)

(21) Appl. No.: 19/658,776

(22) Filed: Apr. 27, 2026

Related U.S. Application Data
(60) Provisional application No. 63/796,162, filed on Apr. 28, 2025.

(51) Int. Cl. A61K 31/675; A61K 9/00; A61K 9/08; A61K 9/16; A61P 31/18

(52) U.S. Cl. CPC A61K 31/675; A61K 9/0031; A61K 9/08; A61K 9/1611; A61P 31/18

(57) ABSTRACT

Prophylactic dosing regimens to administer hypo-osmolar compositions of tenofovir or derivatives thereof into the rectum of a subject to provide long-lasting protection against infection by HIV via receptive sexual intercourse (RSI) are provided. The regimens prevent infection or replication of HIV in the intestines of a subject for a period of 96 hours or more. The regimens include intra-rectal administration of a hypo-osmolar solution of tenofovir or a derivative thereof as two discrete applications, separated in time, e.g., by 60 minutes or more or less, preferably no more than 10 minutes. Each application includes a hypo-osmolar aqueous solution of the tenofovir or derivative thereof (i.e., approximately 600 and 700 mg of the tenofovir or derivative thereof in a volume of between approximately 100 ml and 150 ml, inclusive).

### CROSS-REFERENCE TO RELATED APPLICATIONS

This application claims the benefit of and priority to U.S. Provisional Application No. 63/796,162 filed on Apr. 28, 2025, the contents of which is incorporated herein in its entirety.

### STATEMENT REGARDING FEDERALLY SPONSORED RESEARCH OR DEVELOPMENT

This invention was made with government support under grant AI113127 awarded by the National Institutes of Health. The government has certain rights in the invention.

### BACKGROUND OF THE INVENTION

HIV and STIs are epidemic in populations of individuals at increased risk of infection. In an effort to expand pre-exposure prophylaxis (PrEP) options to include an on-demand, non-systemic, topical, behaviorally congruent option for those with substantial risk of HIV acquisition through condomless receptive sexual intercourse (RSI), such as receptive anal intercourse (RAI) and/or receptive vaginal intercourse (RVI), global HIV PrEP options for preventing sexual HIV acquisition now include oral daily emtricitabine/tenofovir disoproxil fumarate (F/TDF) and emtricitabine/tenofovir alafenamide (F/TAF) tablets, monthly dapivirine vaginal ring (DPV VR) and intramuscular injectable bi-monthly cabotegravir (CAB-LA).

All of these medical interventions can be efficacious when taken as directed. However, all modalities can fail to prevent HIV acquisition and may be associated with HIV drug resistance when doses are missed, and when compared to female contraceptive formulation options, both in variety of drugs and variety of dosing formulation types, the options for PrEP are meager. In addition, uptake of PrEP, even in well-resourced healthcare systems, has been low. In the US, it is estimated that only <25% of persons who would benefit from PrEP are currently taking PrEP. Cost, availability, awareness, personal sense of risk, and provider willingness are commonly cited barriers for uptake. Efforts to develop the vaginal ring and injectable PrEP after the efficacy of oral F/TDF was proven effective were focused, in part, on expanding options to include longer-acting formulations and simplifying daily oral F/TDF or F/TAF PrEP regimens. Monthly vaginal rings or bi-monthly intramuscular injections require fewer doses per unit time. However, both have limitations as well. CAB-LA injections are associated with a high frequency of moderately painful injection site reactions and systemic drug concentrations sustained a year or longer after cessation of dosing. The DPV VR avoids systemic drug concentrations of any pharmacologic consequence, but the VR may be apparent to partners and, even with very high levels of adherence, does not provide the same level of protection as oral F/TDF or CAB-LA and does not provide protection from RAI and/or RVI.

RAI and/or RVI remains a substantial risk for HIV acquisition globally. RAI is the principal HIV risk for cisgender men and transgender women who have sex with men (MSM and TGW), who together include 69% of US (2018) and 52% of global (2019) HIV infections annually; further, the attributable fractions of these populations for condomless or unprotected RAI (URAI) is estimated to be 64%. Men who have sex with men are at high risk of HIV acquisition through unprotected RAI or RVI, and non-systemic (e.g., rectal), on demand, behaviorally-congruent HIV PrEP has long been advocated by individuals who find adherence challenging or prefer minimizing systemic drug concentrations.

As URAI carries a greater than 10-fold greater likelihood of HIV acquisition per exposure than unprotected receptive vaginal intercourse (URVI), even given far less frequent URAI than URVI in women, the global population attributable risk of HIV acquisition from RAI in women has been estimated at ~40%.

To date, there are no PrEP options for protection from HIV acquisition from RAI that are on-demand, non-systemic, and behaviorally congruent, i.e., consistent with an existing practice or behavior of sex-related product use.

Given the (1) very high efficacy of TFV-based PrEP, (2) more efficient delivery of active drug to the colon with rectal compared to oral dosing, (3) the large negative impact of poor adherence on PrEP outcomes, (4) frequency and impact of URAI in men and women with regard to HIV acquisition, and (5) the common pre-existing behavior of enemas use before and after RAI and RVI by many MSM and female sex worker populations, respectively, there exists an unmet need for the development of a pharmacokinetically-enhanced TFV prodrug enema capable of protecting subjects from HIV acquisition.

It is therefore an object of the present invention to provide regimens for administration rectally to deliver an effective amount of antimicrobial, especially antiviral, to reduce the likelihood of HIV infection.

It is a further object of the present invention to provide such methods and formulations which are safe, comfortable and easy to store and use.

### SUMMARY OF THE INVENTION

Dosing regimens for administering hypo-osmolar compositions of tenofovir and derivatives thereof into the rectum of a subject to provide long-lasting protection to the subject against infection by Human Immunodeficiency Virus (HIV) via receptive anal intercourse (RAI) have been developed. Preferably, the administration regimen prevents infection or replication of HIV in the intestines of a subject for a period of at least 120, up to 168 hours (seven days), or more following administration. Prophylactic dosing regimens include intra-rectal administration of a hypo-osmolar solution of tenofovir or a derivative thereof as two discrete applications, separated in time by no more than 60 minutes, preferably no more than 10 minutes. Each application introduces into the rectal cavity a hypo-osmolar aqueous solution of tenofovir or a derivative thereof (i.e., approximately 600 and 700 mg of the tenofovir or derivative thereof in a volume of between approximately 100 ml and 150 ml, inclusive). The formulations may include additional agents such as buffers, or preservatives. The desired hypo-osmolality is established by managing the NaCl content and varies with the amount of TFV in the solution. In some forms, additional agents such as buffers (e.g., NaOH for pH control), salts (e.g., NaCl for osmolality adjustment), and polymers (within vaginal formulations) are added to optimize desired pharmaceutical properties.

The formulations are preferably stored at room temperature and administered rectally or vaginally for at least 20 minutes. Up to one hour prior to the initiation of RAI or RVI, respectively

A dosage form of tenofovir or a derivative thereof, including a prophylactically effective amount to inhibit HIV infection in a subject for a period of at least 96 hours following rectal administration, is provided.

In some forms, the dosage form includes tenofovir is 9-[9 (R)-2-(phosphonomethoxy)propyl] adenine (PMPA). In some forms the tenofovir derivative is tenofovir disoproxil fumarate (TDF), tenofovir exalidex or tenofovir alafenamide (TAF). In some forms the tenofovir or derivative thereof is in an amount of between about 1,000 mg and about 1,500 mg, inclusive. In some forms, the tenofovir or derivative thereof is in an amount of about 1,320 mg. In some forms, the dosage form includes one or more of NaCl, NaOH, or NaHCO₃. In some forms, the final concentration of sodium in the dosage form is between about 0.4% and about 0.5%, inclusive.

In some forms, the tenofovir or derivative thereof is in the form of a solid, a liquid, or a semi-solid. Exemplary solid forms include granules, a powder, a tablet, and lyophilized or dried material. An exemplary powder includes particles of between about 20 μm to about 600 μm in diameter, inclusive. In specific forms, the dosage form includes an amount of a solid form of tenofovir or derivative thereof and an amount of sodium chloride to yield, when reconstituted as an aqueous solution, a hypotonic solution of from 145 mOsm/kg to about 290 mOsm/kg including from 0.1 mg/ml to 20 mg/ml, 1.8 mg/ml to 10 mg/ml, 2 mg/ml to 10 mg/ml, or 5.28 mg/ml, of tenofovir or a therapeutic or molar equivalent amount of a derivative thereof. Exemplary containers include a cylindrical profile, optionally including a polyester, such as a biaxially oriented polyethylene terephthalate, such as a metallized biaxially-oriented polyethylene terephthalate. Exemplary dosage forms include a sterile, lyophilized powder in an amount of about 1,320 mg, packaged within a hermetically sealed container. In some forms, a hermetically sealed container includes two compartments, each including approximately 660 mg of the powder. In some forms the container includes a stick pack including one or more tear notch at one or more end(s), or a sachet including one or more tear notch at one or more end(s), for example, wherein a tear notch provides an opening to a single compartment of the container.

Methods for preparing a dosage form of tenofovir or a derivative thereof are also provided. In some forms, the methods include: (a) dissolving tenofovir or a derivative thereof in water to provide a solution; (b) adjusting the pH of the solution to a pH ranging from 6.0 to 8.0 or from 6.5 to 7.5; (c) spray drying the solution under conditions for producing a powder including the tenofovir or a derivative thereof; and (d) mixing the tenofovir or a derivative with a salt or buffer in amounts to produce, when dissolved in water to a volume of between about 240 mL and about 260 mL, inclusive, a prophylactically-effective concentration of the tenofovir or a derivative thereof with an osmolality of about 145±22 milliosmoles per kilogram of water (mOsm/kg H₂O). In some forms, the methods also include: (e) depositing and/or sealing the tenofovir or a derivative thereof within a container. In some forms, the spray drying is performed under the following conditions: inlet temperature set to about 200 °C, pump flow set to about 4 mL/min, aspirator set to about 35 m3/h, and N2 flow set to about 601 L/h.

Methods for administration of the dosage forms of tenofovir or a derivative thereof are also provided. In some forms, the methods include administering into the rectum of a subject the effective amount of tenofovir or a derivative thereof to inhibit infection or replication of HIV in the rectum and/or intestine of the subject for a period of at least 24 hours, up to 96 hours following administration, where the tenofovir or a derivative thereof is administered into the rectum in the form of a hypo-osmotic aqueous solution, and wherein the aqueous solution is applied into the rectum as a first and a second discrete applications, and where the first and second discrete applications are separated by administration of one or more further solutions into the rectum. In some forms the aqueous solution of the first and/or second discrete applications is retained within the rectum for a period of between about 10 seconds and about 10 minutes, inclusive. In some forms each of the first and second discrete applications into the rectum includes administering approximately 50% of the amount of tenofovir or a derivative thereof. In some forms each of the first and second discrete applications into the rectum includes between about 100 ml and about 150 ml, inclusive, of the aqueous solution. In some forms, the methods including one or more discrete application of a further solution into the rectum after the first and before the second discrete applications. Exemplary solutions include water, an aqueous solution of tenofovir or a derivative thereof, saline, and other aqueous solutions. In some forms, administration of the dosage form is repeated once or more than once. For example, in some the administration is repeated 4 days after the first administration. In some forms, the administration is repeated not more than twice a week.

Methods for reducing or preventing HIV infection associated with receptive sexual intercourse (RSI) in a subject are also provided. Typically, the methods include: (i) administering into the sex passage of the subject at least 20 minutes, up to 96 hours prior to RSI a first solution including between 600 and 700 mg inclusive of tenofovir or a derivative thereof in a volume of between about 100 ml and about 150 ml, inclusive; (ii) optionally retaining the solution within the sex passage for a period of between about 10 seconds and about 10 minutes, inclusive; (iii) draining/defecating the solution from the sex passage; and (iv) administering into the sex passage of the subject at least 20 minutes, up to 60 minutes following RSI a second solution including between 600 and 700 mg inclusive of tenofovir or a derivative thereof in a volume of between about 100 ml and about 150 ml, inclusive. In some forms, one or more further solution(s) in an amount between about 1 ml and 500 ml is administered into the sex passage of the subject following RAI and prior to administering the second solution in (iv). In some forms, the one or more further solution(s) does not include tenofovir or a derivative thereof.

In some forms, the methods further include: (v) repeating (i)-(iv) after a period of 24 to 96 hours. In some forms, the method is repeated no more than once in a week. Kits of parts include the described dosage form of tenofovir or a derivative thereof and one or more of (i) instructions for use according to the described methods of use; and/or (ii) a container for administration as an enema; and/or (iii) an aqueous solution are also provided.

### BRIEF DESCRIPTION OF THE DRAWINGS

FIGS. 1A-1B are graphs of the tissue cell MMC TFV-DP (fmol/10⁶ cells) (FIG. 1A) and plasma TFV (ng/mL) (FIG. 1B), respectively, showing the log 10 concentration-time plots for DREAM-01 and DREAM-03 Product C (660 mg TFV in 125 mL half-normal saline) only (including TFV once (●); TFV-TFV-TFV (▪) TFV-H2O—H2O (▾); and H2O—H2O-TFV (▴)) over time (intervals of 0-6, and 12-168 Hours). Model-based simulation of oral on demand “2-1-1” TDF regimens (steady-state [SS] and first use of the regimen [1st]) are added for reference (dotted lines).

FIG. 2 is a graph of data from a colorectal tissue ex vivo HIV challenge, showing log 10 weight-adjusted p24 antigen (y-axis) versus rectal tissue homogenate TFV-DP (x-axis), showing plot points for DREAM-01 TFV douche (circles) contrasted to each of several MTN-017 dosing regimens, including daily oral (diamond), daily TFV RGVF gel (diamond), on-demand RAI-associated TFV reduced glycerin vaginal formulation (RGVF) rectal gel (diamond), respectively. Baseline values are imputed. Imax model is based upon the fit of DREAM-01 data only (solid line).

FIG. 3 is a schematic depicting the timeline of methods and sample collection for a study to determine the optimal sequence of dosing relative to RAI (e.g., TFV douching before vs. after RAI). Initial screening is carried out prior to Sequence A; a pre-A screen is followed by a TVF douche before RAI (according to Sequence A; tenofovir rectal douche once and evacuation followed by simulated RAI.); and a pre-B screen is followed by a TVF douche after RAI (according to Sequence B; simulated RAI followed by tenofovir rectal douche and evacuation). Timing (in minutes) for each Sequence is depicted for each of TVF douche, RAI with SF, Blood screen, Seminal Fluid screen and PSPECT/CT, respectively. †Pre-B phase was also a washout phase that was ≥14 days. * Baseline blood samples were only obtained for participants on oral PrEP within 30 minutes.

FIG. 4 is a graph of the intraluminal colonic distribution of radiotracers in each participant from the study, including Dₘᵢₙ (most distal extent of signal, lower error bar), Dₘₐₓ (maximal proximal extent, upper error bar), and Dₐᵥₑ (mean residence distance, closed circle), for each of Sequences A and B, respectively, for each of the two radiotracers (¹¹¹In-DPTA (“A”); and ^{99m}Tc-Sc (“B”)), respectively. The anorectal junction (ARJ) was used as the “0” reference point.

FIG. 5 is a graph of Plasma TFV during the first hour after dosing for each of Sequences A ( [symbol] ) and B ( [symbol] ), respectively, from the study in FIG. 3. Data are represented as median (range). N=4 for each sequence.

### DETAILED DESCRIPTION OF THE INVENTION

### A. Definitions

As generally used herein “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

“Biocompatible” and “biologically compatible”, as used herein, generally refer to materials that are, along with any metabolites or degradation products thereof, generally non-toxic to the recipient, and do not cause any significant adverse effects to the recipient. Generally speaking, biocompatible materials are materials which do not elicit a significant inflammatory, immune or toxic response when administered to an individual.

The terms “gel” and “hydrogel”, as used interchangeably herein, refer to a swollen, water-containing network of finely-dispersed polymer chains that are water-insoluble, where the polymeric molecules are in the external or dispersion phase and water (or an aqueous solution) forms the internal or dispersed phase. The chains can be chemically crosslinked (chemical gels) or physically crosslinked (physical gels). Chemical gels possess polymer chains that are connected through covalent bonds, whereas physical gels have polymer chains linked by non-covalent bonds or cohesion forces, such as Van der Waals interactions, ionic interaction, hydrogen bonding, or hydrophobic interaction.

“Hydrophilic,” as used herein, refers to molecules which have a greater affinity for, and thus solubility in, water as compared to organic solvents. The hydrophilicity of a compound can be quantified by measuring its partition coefficient between water (or a buffered aqueous solution) and a water-immiscible organic solvent, such as octanol, ethyl acetate, methylene chloride, or methyl tert-butyl ether. If after equilibration a greater concentration of the compound is present in the water than in the organic solvent, then the compound is considered hydrophilic.

“Hydrophobic,” as used herein, refers to molecules which have a greater affinity for, and thus solubility in, organic solvents as compared to water. The hydrophobicity of a compound can be quantified by measuring its partition coefficient between water (or a buffered aqueous solution) and a water-immiscible organic solvent, such as octanol, ethyl acetate, methylene chloride, or methyl tert-butyl ether. If after equilibration a greater concentration of the compound is present in the organic solvent than in the water, then the compound is considered hydrophobic.

As used herein, the term “treating” includes inhibiting, alleviating, preventing or eliminating one or more symptoms or side effects associated with the disease, condition, or disorder being treated.

The term “reduce”, “inhibit”, “alleviate” or “decrease” are used relative to a control. One of skill in the art would readily identify the appropriate control to use for each experiment. For example, a decreased response in a subject or cell treated with a compound is compared to a response in subject or cell that is not treated with the compound.

As used herein the term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease or disorder, and the treatment being administered. The effect of the effective amount can be relative to a control. Such controls are known in the art and discussed herein, and can be, for example the condition of the subject prior to or in the absence of administration of the drug, or drug combination, or in the case of drug combinations, the effect of the combination can be compared to the effect of administration of only one of the drugs.

“Excipient” is used herein to include any other compound that can be included in the formulation that is not a therapeutically or biologically active compound. As such, an excipient should be pharmaceutically or biologically acceptable and non-toxic to the subject when administered by the intended route.

The term “osmolarity”, as generally used herein, refers to the total number of dissolved components per liter. Osmolarity is similar to molarity but includes the total number of moles of dissolved species in solution. An osmolarity of 1 Osm/L means there is 1 mole of dissolved components per L of solution. Some solutes, such as ionic solutes that dissociate in solution, will contribute more than 1 mole of dissolved components per mole of solute in the solution. For example, NaCl dissociates into Na+ and Cl— in solution and thus provides 2 moles of dissolved components per 1 mole of dissolved NaCl in solution. Physiological osmolarity is typically in the range of about 280 to about 310 mOsm/L.

The term “hypo-osmolar”, as generally used herein, refers to a solution having a lower total concentration of solutes compared to human plasma or to the cytoplasm of a cell, i.e., below about 250-275 mOsm/kg. As used herein, the term “mildly hypoosmotic” means an aqueous solution or carrier having an osmolarity greater than tap water (0 mOsm), and between about 20 mOsm to about 290 mOsm.

The “effective amount” is safe, at least to the extent the benefits of treatment outweigh the detriments, and/or the detriments are acceptable to one of ordinary skill and/or to an appropriate regulatory agency, such as the U.S. Food and Drug Administration.

A therapeutically effective amount of an active agent may vary according to factors such as the disease state, age, sex, and weight of the individual, and the ability of the active agent to elicit a desired response in the individual. A “prophylactically effective amount” refers to an amount effective, at dosages and for periods of time necessary, to achieve a desired prophylactic result. Typically, since a prophylactic dose is used in subjects prior to or at an earlier stage of disease, the prophylactically effective amount may be less than the therapeutically effective amount.

The term “tonicity”, as generally used herein, refers to the osmotic pressure gradient resulting from the separation of two solutions by a semi-permeable membrane. In particular, tonicity is used to describe the osmotic pressure created across a cell membrane when a cell is exposed to an external solution. Solutes that readily cross the cellular membrane contribute minimally to the final osmotic pressure gradient. In contrast, those dissolved species that do not cross the cell membrane, “impermeable solutes”, will contribute to osmotic pressure differences and thus tonicity.

The term “hypertonic”, as generally used herein, refers to a solution with a higher concentration of impermeable solutes than is present on the inside of the cell. When a cell is immersed into a hypertonic solution, water will flow out of the cell, concentrating the impermeable solutes inside the cell until it becomes equal to the concentration of impermeable solutes outside the cell.

The term “hypotonic”, as generally used herein, refers to a solution with a lower concentration of impermeable solutes than is present inside of the cell. When a cell is immersed into a hypotonic solution, water will flow into the cell, diluting the concentration of impermeable solutes inside the cell until it becomes equal to the concentration of impermeable solutes outside the cell.

The term “isotonic”, as generally used herein, refers to a solution wherein the osmotic pressure gradient across the cell membrane is balanced and no water flows into or out of the cell. The same meanings for tonicity apply for water flow through intestinal epithelia; hypertonic solutions cause water to flow into the lumen, whereas hypotonic solutions cause water to flow out of the lumen. Tonicity depends on the permeability properties of the cell or epithelium to different solutes, whereas osmolarity depends only on the total concentration of all solutes.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### B. Methods for Prophylactic or Post-Exposure Protection from HIV Infection

It has been established that specific regimes for administering hypo-osmolar compositions of tenofovir and derivatives thereof into the rectum of a subject can provide protection to the subject against infection by Human Immunodeficiency Virus (HIV) via receptive anal intercourse (RAI) based upon clinical pharmacokinetic results compared to in vitro IC50 values and ex vivo HIV challenge of colon tissue after in vivo (clinical) dosing). In addition, efficacy of a rectal tenofovir douche has been demonstrated in a macaque SHIV rectal challenge model. Preferably, the administration regimen prevents infection or replication of HIV in the intestines of a subject for a period of 24 hours, up to 120 hours, or up to 168 hours (seven days), or more. Typically, the regimens include intra-rectal administration of a hypo-osmolar solution of tenofovir or a derivative thereof as two discrete applications, separated in time by no more than 60 minutes, preferably no more than 10 minutes. Each application includes a hypo-osmolar aqueous solution of the tenofovir or derivative thereof (i.e., approximately 600 and 700 mg of the tenofovir or derivative thereof in a volume of between approximately 100 ml and 150 ml, inclusive). In an exemplary form, the methods administer hypo-osmolar aqueous solutions of tenofovir as a series of two rectal douches that are directly applied via the anus into the rectal cavity. In some forms, the methods include retaining the douche within the rectal cavity for a period of time between about 1 second and about 10 minutes, inclusive, such as between about 1 minute and about 3 minutes, inclusive. In some forms, the methods incorporate application of one or more additional “wash” solutions between the first and second application of the tenofovir or derivative thereof.

Therefore, in some forms, method to protect a subject from infection with HIV via RAI include administering an effective amount of a hypo-osmolar aqueous solution of tenofovir or a derivative thereof into the rectum of the subject to inhibit infection or replication of HIV in the rectum and/or intestine of the subject for a period of at least 168 hours (7 days) following administration. Typically, the methods administer the hypo-osmolar composition of tenofovir or derivative thereof into the rectum as a first and a second discrete applications, where each of the first and second discrete applications are separated in time by less than about 20 minutes. Typically, each of the first and second discrete applications into the rectum include administering approximately 50% of the effective amount of tenofovir or a derivative thereof. In some forms, one or more discrete application of a further solution into the rectum of the subject occurs after the first and before the second discrete applications. In some forms, the further solution is selected from water, an aqueous solution of tenofovir or a derivative thereof, saline, and other aqueous solutions.

Dosage regimens may be adjusted to provide an optimum desired response (e.g., prophylactic response), and/or to maintain desirable therapeutic and/or prophylactic levels of an active agent. In non-limiting forms, the present devices, kits, and methods provide a desirable level of active agent for up to 120 hours, or 168 hours in vivo. For example, a single dose or bolus may be administered, several divided doses may be administered over time, or the composition may be administered continuously or in a pulsed fashion with doses or partial doses being administered at regular intervals.

In some forms, a single TFV douche achieves rectal tissue concentrations effective to provide protection equal or superior to oral dosing of tenofovir and emtricitabine in a fixed dose combination. In some forms, rectal application of a single tenofovir douche that precedes and follows an unlimited number of non-medicated, cleansing water or NaCl solutions administered rectally in between will provide a high level of protection similar to a single tenofovir medicated douche without any subsequent cleansing douches. In some forms, a post-RAI douche is administered, but is not followed by any further non-medicated douches to ensure high concentrations sufficient for protection against HIV infection.

### 1. Dosing Regimens

Dosing regimens that enhance the efficacy of antiviral prophylactic and post-exposure treatments for the prevention of HIV transmission associated with RSI (RAI and/or RVI) are provided. The dosing regimens are designed for integration with existing practices that are known to be commonly associated with RAI. As described in the Examples, assessments of study participants who engage in RAI identified rectal douching prior to and/or immediately subsequent to RAI as a commonly adopted practice, for example, amongst men who have sex with men (MSM). Therefore, the dosing regimens are designed to integrate within existing practices for RAI and/or RVI to achieve maximal compliance for HIV control/prevention.

Rectal douching was identified as a commonly adopted practice, with more than one pre-RAI douche applications being most commonly adopted, and/or commonly at least one post-RAI application. Therefore, the dosing regimens include administering an effective amount of tenofovir or a derivative thereof as a solution for intrarectal administration as a first and second applications, separated in time by less than one hour, prior to or immediately subsequent to RAI and/or RVI.

Typically, a dosing regimen includes administering to a subject prior to RAI a first rectal douche as a first application containing between approximately 600 and 700 mg of tenofovir or derivative thereof in a volume between about approximately 100 ml and 150 ml, inclusive; optionally retaining the first rectal douche for a period of between about 10 seconds up to about 10 minutes; and removing/defecating the first rectal douche from the rectal cavity. The dosing regimen provides a rectum tissue drug concentration sufficient to protect the subject from HIV infection resulting from the RAI. In some forms, the methods further include administering to the subject a second rectal douche as a second application containing between approximately 600 mg and 700 mg of tenofovir or derivative thereof in a volume between about approximately 100 ml and 150 ml, inclusive; optionally retaining the second rectal douche for a period of between about 10 seconds, up to 10 minutes, inclusive; and removing/defecating the second rectal douche from the rectal cavity.

In some forms, the dosing regimens are carried out prior to RAI by a subject, such as immediately prior to RAI or RVI, or from 20 minutes to one hour, up to 24 hours prior to RAI. In other forms, the dosing regimens are carried out subsequent to RAI or RVI by a subject, such as immediately after RAI or RVI, or one, two, three, four, five, six, or seven minutes, up to ten minutes subsequent to RAI. In all regimens, it is important that the dosing regimen is completed within 1, 5, 10, 15, 30, or 45 minutes, or 1 hour prior to a potential exposure to HIV via RAI or RVI. It has been established that douching after RAI or RVI with a non-medicated douche will potentially reduce the concentration of TFV in tissue and passages (rectum or vagina, respectively) and increase the distribution of HIV in the passage. Therefore, in some forms, when a subject rectally administers a second of further douche for any reason, e.g., cleansing, after RAI, the second or further douche is medicated with TFV. Therefore, in some forms, when a subject vaginally administers a second of further douche for any reason, e.g., cleansing, after RVI, the second or further douche is medicated with TFV.

i. Optional Wash Steps

Importantly, the efficacy of the methods is not altered by the application of one or more intrarectal “wash” solutions between application of the first and second solutions of between approximately 600 and 700 mg of tenofovir or derivative thereof. As set forth in the Examples, it has been established that duplicated intrarectal administration of between about 600 and 700 mg TFV induces a sufficient initial “peak” plasma concentration to prevent HIV infection and/or replication throughout the rectosigmoid colon, which persists for up to 5, 6 or 7 days, provided that the final application is permitted to persist within the rectal cavity following application without additional non-medicated douches. Therefore, for persons who typically douche only once before RAI, the described dosing regimens require at least application of one solution of between approximately 600 mg and 700 mg of tenofovir or derivative thereof. For persons typically douching with more than one douche (~100 to 150 mL equivalent), the regimen requires rectally administering a first solution and a second solution of between approximately 600 mg and 700 mg of tenofovir or derivative thereof, whereby the second application may be preceded by one or more non-medicated douches, but is not followed by a subsequent “wash” solution for at least another 24 hours. For persons who typically douche after RAI, an additional medicated TFV douche may be administered after RAI without any non-medicated douches administered thereafter for a period of at least 24 hours.

As set forth in the Examples, it has been determined that subjects who apply a rectal douche prior to and/or after RAI typically apply more than two douche solutions (e.g., 3-5) into the rectum in a step-wise manner, for example, to cleanse and/or lubricate. Therefore, in some forms, the methods further include intrarectal application of one or more additional douche solutions, for example, after administering the first intrarectal hypo-osmolar solution of between approximately 600 and 700 mg of tenofovir or derivative thereof, and prior to administering the second intrarectal hypo-osmolar solution of between approximately 600 and 700 mg of tenofovir or derivative thereof. For example, in some forms, the methods include administering one or more intrarectal “wash” solutions, such as an intrarectal aqueous solution, after the first intrarectal hypo-osmolar solution of between approximately 600 and 700 mg of tenofovir or derivative thereof, and prior to applying the second intrarectal hypo-osmolar solution of between approximately 600 and 700 mg of tenofovir or derivative thereof. In some forms, the methods administer from one to ten, such as 1, 2, 3, 4, 5, 6, 7, 8, 9 or 10 discrete intrarectal applications of a solution as “wash” solutions, prior to applying the second intrarectal hypo-osmolar solution of between approximately 600 and 700 mg of tenofovir or derivative thereof.

In some forms, each of the “wash” solutions include an active agent, such as tenofovir or a derivative thereof. For example, in some forms, one or more wash solutions includes an intrarectal hypo-osmolar solution of between approximately 600 and 700 mg of tenofovir or a derivative thereof. In other forms, one or more of the “wash” solutions includes an aqueous solution, such as water, or saline, in the absence of an additional active agent.

ii. Formulating Solutions for Intrarectal Administration

Any of the described methods can include one or more steps for formulating one or more of the intrarectal compositions required for the methods.

The formulations are typically prepared and administered as described in the Examples. Typically, the formulations are prepared as pre-solubilized drug which is provided in a container, such as a single use, squeezable container with a flexible tip for ease of rectal insertion, for example, between one and 24 hours prior to RAI. In some forms, a formulation is also administered soon after intercourse. In some forms, a formulation is administered immediately prior to RAI, although this is not preferred.

In some forms, a formulation including tenofovir or a derivative thereof is provided as a tablet or as powder or granules from a sachet which is added to a reusable or single use container, to which water is also added to dissolve the tablet, granules, or powder. In some forms, the tablet, powder, or granules contains salts, and/or a gel forming polymer as well as the tenofovir or a derivative thereof and any other additives.

For rectal administration, it may be preferred to use an applicator which distributes the composition substantially evenly throughout the rectum. For example, a suitable applicator is a tube 2.5 to 25 cm, preferably 5 to 10 cm, in length having holes distributed regularly along its length which is capable of distribution of the formulations within the colon.

The terms “reduce”, “suppress” and “inhibit,” as well as words stemming therefrom, have their commonly understood meaning of lessening or decreasing. These words do not necessarily imply 100% or complete treatment, reduction, suppression, or inhibition.

In some forms, compositions including hypo-osmolar solutions of tenofovir or a derivative thereof are administered to the rectum of the subject at least 30 minutes to 20 minutes before RAI. In other forms, the microbicidal compositions are also administered to the rectum of the subject at least 30 minutes to 20 minutes after RAI. The formulations may provide inhibition of HIV infection of the colon and rectum for up to 7 days post application with one or several doses.

Patterns of use of enemas underline the importance of the rapid achievement of sustained drug levels in the relevant tissues. Modeling studies indicate oral TFV PrEP regimens require one week to achieve protective concentrations of the active drug form, TFV diphosphate (TFV-DP), in tissue. These concentrations can be achieved within 30 minutes after TFV 1% rectal gel dosing. Another marked advantage of topical strategies is the limited systemic absorption of drug, which minimizes adverse long- and short-term systemic side effects.

iii. Additional Treatments

In some forms, additional treatments or reagents are administered to the subject together with the described dosage regimens for TFVs. Exemplary additional therapeutic compositions/active can include, but are not limited to, antiretroviral compositions (e.g., nucleoside reverse transcriptase inhibitors, nonnucleoside reverse transcriptase inhibitors, protease inhibitors, fusion inhibitors, entry inhibitors, and integrase strand transfer inhibitors, such as, for example and without limitation, efavirenz, emtricitabine, rilpivirine, atazanavir sulfate, darunavir ethanolate, elvitegravir, lamivudine, zidovudine, abacavir, zalcitabine, dideoxycytidine, azidothymidine, didanosine, dideoxyinosine, stavudine, rilpivirine, etravirine, delvaridine, nevirapine, doravirine, amprenavir, tipranavir, indinavir, saquinavir, lopinavir, ritonavir, fosamprenavir, ritonavir, darunavir, atazanavir, nelfinavir, enfuvirtide, raltegravir, dolutegravir, elvitegravir, cabotegravir, raltegravir, islatravir, maraviroc, DS003, antiviral compositions (e.g., 4′-Ethynyl-2-fluoro-2′-deoxyadenosine (EFdA, islatravir) and MK-8527), nucleoside analogs, such as: acyclovir (2-amino-9-(2-hydroxyethoxymethyl)-3H-purin-6-one), penciclovir (2-amino-9-[4-hydroxy-3-(hydroxymethyl)butyl]-3H-purin-6-one), foscarnet (phosphonoformic acid), cidofovir ([(2S)-1-(4-amino-2-oxopyrimidin-1-yl)-3-hydroxypropan-2-yl]oxymethylphosphonic acid), adefovir (2-(6-aminopurin-9-yl) ethoxymethylphosphonic acid), and pharmaceutically-acceptable ester prodrugs thereof, such as valaciclovir (valine aciclovir ester, 2-[(2-amino-6-oxo-3H-purin-9-yl) methoxy]ethyl(2S)-2-amino-3-methylbutanoate) or famciclovir ([2-(acetyloxymethyl)-4-(2-aminopurin-9-yl)butyl] acetate)), antibiotic/antiprotozoal compositions (e.g., GRFT, CSIC, metronidazole), antifungal compositions (e.g., clotrimazole), hormones or hormonal compositions (e.g., levonorgestrel, etonogrestrel, desogrestrel, dienogest), hormonal receptor modulators (e.g., ulipristal acetate), antibacterial compounds, e.g., compounds active against bacterial sexually transmistted vaignal and rectal infections, like doxycycline), and bases or salts of the foregoing. In addition, other compounds such as RANTES derivatives and retrocyclin (e.g., RC-IO I) can be included. Compositions that affect metabolism of another composition, such as antiretroviral compositions, such as cobicistat (sold under the trade name TYBOOST®) can also be included. For example, atazanavir and cobicistat (sold under the trade name EVOTAZ®).

iv. Exemplary Dosing Regimen

An exemplary dosing regimen for protection of a subject from infection by HIV via receptive anal intercourse (RAI) includes:
(a) administering into the rectum of the subject a first composition including a hypo-osmolar solution containing approximately 660 mg of tenofovir or a derivative thereof (TFV), in a volume of approximately 125 ml;
(b) optionally retaining the first composition within the rectal cavity for a period of 10 seconds up to 10 minutes, preferably less than one minute;
(c) removing/defecating the first composition from the rectal cavity;
(d) optionally washing the rectal cavity by administering one or more intrarectal wash solutions into the rectal cavity and removing/defecating the one or more intrarectal wash solutions;
(e) administering a second composition including a hypo-osmolar solution containing approximately 660 mg of TFV in a volume of approximately 125 ml; and
(f) optionally retaining the second composition within the rectal cavity for a period of 10 seconds up to 10 minutes, preferably less than one minute.

In preferred forms, the dosing regimen is completed at one hour, up to 24 hours prior to RAI, or within 10 minutes subsequent to RAI, or both.

In some forms, the dosing regimen is repeated up to four days following the first administration. For example, if RAI is anticipated within 4 days following a prior administration, there is no need for a second administration. However, if RAI is anticipated more than 4 days after a first administration, in some forms the dosage regimen is administered every 4 days. In some forms, the dosage regimen is administered no more than twice a week.

An exemplary subject is an adult human who is at risk or exposure to HIV via RAI. Typically, the subject is HIV sero-negative.

### C. Compositions of Tenofovir or Derivatives Thereof

It has been established that hypo-osmolar formulations including an effective amount of tenofovir or a derivative thereof, administered to a subject as a rectal solution in a regimen of at least two discrete applications. e.g., separated in time by less than 60 minutes, preferably less than 10 minutes, prevents infection or replication of HIV within the intestine of the subject for a period of at least 4, up to 7 days. Accordingly, dosage formulations of tenofovir or a derivative thereof are provided for intrarectal administration. In some forms, the formulations include a hyo-osmolar solution that is administered to a subject in two discrete rectal applications, for example, as two discrete solutions of approximately 40%-60% of the volume of the adult human rectal cavity, or a volume of between about 100 ml and about 150 ml, inclusive.

Typically, the dosage forms include intrarectal hypo-osmolar aqueous solutions of tenofovir or a derivative thereof. Suitable dosage forms include single-dose, or multiple-dose sachets, vials, or other containers for dry-powder drug products. The dry powder drug product may be reconstituted in any useful container for ultimate delivery in liquid form as an enema or douche. Suitable containers for delivery include, without limitation, an enema bag, an enema bottle, an enema bottle having an extended tip, medical syringes or droppers, containing a composition including an active ingredient useful for PrEP. Additional dosage forms may include a rectal dosage form configured in a liquid dosage form, a solid dosage form, or a semi-solid dosage form.

The therapeutic compositions may be sterile and should be stable under the conditions of manufacture and storage. For example, sterile powders can be prepared by incorporating the active agent in an appropriate solvent with one, or a combination of ingredients, followed by filter-sterilization. The liquid may be spray-dried, vacuum dried, lyophilized, or otherwise dried, under aseptic conditions, and packaging, such as sachets or other containers, may be sterilized prior to filling with the powder described herein under aseptic conditions. Once filled, the sachet or other container may be further sterilized, e.g., in a manner that does not, or does not substantially deteriorate the powdered drug product within a sachet or other container.

### 1. Tenofovir and Pro-drugs or Derivatives

Tenofovir, also known as 9-R-[2-(phosphonomethoxypropyl) adenine] or PMPA, is an antiretroviral drug used to treat HIV and hepatitis B, and is a nucleotide analog reverse transcriptase inhibitor (NtRTI) that inhibits HIV reverse transcriptase and hepatitis B virus polymerase. Oral formulations use prodrugs for enhanced oral absorption.

Tenofovir (PMPA; CAS Number: 147127-20-6) has a molecular formula C₉H₁₄N₅O₄P, and a molecular mass of 287.21 g/mol, and a structure as set forth below:

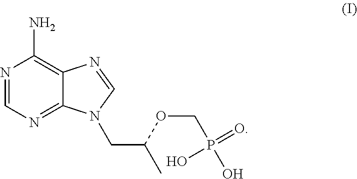 [SMILES: C[C@H](Cn1cnc2c(N)ncnc21)OCP(=O)(O)O]

In another form, the tenofovir derivative is tenofovir disoproxil fumarate (2,4,6,8-Tetraoxa-5-phosphanonanedioic acid, 5-[[(1R)-2-(6-amino-9H-purin-9-yl)-1-methylethoxy]methyl]-, bis(1-methylethyl) ester, 5-oxide, (2E)-2-butenedioate (1:1)) which has the following structure:

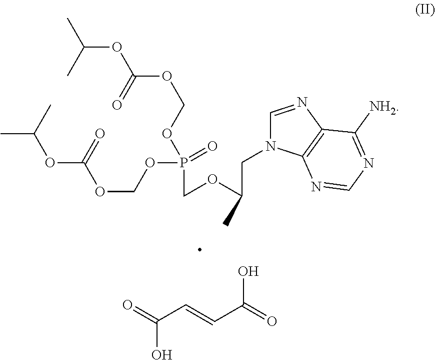 [SMILES: CC(C)OC(=O)OCOP(=O)(CO[C@H](C)Cn1cnc2c(N)ncnc21)OCOC(=O)OC(C)C ; O=C(O)/C=C/C(=O)O]

In some forms, the drug product is in the form of a solid, such as a tablet, powder, granules or a lyophilized material. In some forms, the solid drug product is designed to be reconstituted by an end-user in water to produce a hypoosmotic composition including a prophylactically-effective amount of tenofovir, or a derivative thereof, for example and without limitation ranging from 0.1 mg/ml to 20 mg/ml, 1.8 mg/ml to 10 mg/ml, 2 mg/ml to 10 mg/ml, or 5.28 mg/ml, of tenofovir, tenofovir alafenamide or tenofovir disoproxil fumarate, tenofovir exalidex (CMX-157), or equivalent amounts of another pharmaceutically-effective tenofovir salt.

In some forms, the drug product is provided as a granules or a powder that is reconstituted by an end-user in water to produce a hypoosmotic composition including a prophylactically-effective amount of tenofovir, or a derivative thereof, for example and without limitation ranging from 0.1 mg/ml to 20 mg/ml, 1.8 mg/ml to 10 mg/ml, 2 mg/ml to 10 mg/ml, or 5.28 mg/ml, e.g., of tenofovir, tenofovir alafenamide or tenofovir disoproxil, tenofovir exalidex (CMX-157), or equivalent amounts of another pharmaceutically-effective tenofovir salt.

In some forms, a liquid-dissolved TFV may be spray-dried or lyophilized to produce particles of 20-1000 microns, optionally 20-600 microns, for example less than 50 μm (microns), less than 40 μm, less than 30 μm, less than 25 μm, or less than 20 μm in diameter or particles or less than 50 μm (microns), less than 40 μm, less than 30 μm, less than 25 μm, or less than 20 μm average diameter, as determined by any effective means. In non-limiting forms, the particles, following spray drying, are non-flowing, and have a particle size of less than 20 μm. The dried particles may be subsequently ground or processed with a dry granulation technique together with a salt, buffer, or other excipient (e.g., mannitol or microcrystalline cellulose) to produce larger, flowable particles of, e.g., greater than 50 μm, greater than 60 μm, greater than 75 μm, greater than 90μ, or greater than 100 μm in diameter, or greater than 50 μm, greater than 60 μm, greater than 75 μm, greater than 90 μm, or greater than 100 μm in average diameter, as determined by any effective means. Any useful statistical method may be used to determine average particle size or particle size distribution for a population of particles. Particle size may be measured by, for example and without limitation, sieve analysis, microscopy, light scattering (e.g., dynamic light scattering, laser diffraction, among many other technologies and methods. Particle size analyzers are broadly-available commercially.

In some forms, the drug product is provided as a liquid. For example, in some forms, the drug product is a fully constituted unit dose in an amount of between 100 and 150 ml for administration, for example, dissolved in a suitable buffer and/or packaged in an enema bottle. In some forms, the drug product is a liquid in volume less than 100 ml, such as 10, 20, 30, 40, 50, 60, 70, 80, or 90 ml, having a higher concentration for adding to a full-size unit dose dosing device, for example, by addition of a buffer or water. In some forms, the drug product is a semi-solid gel, or a suppository or a fast-dissolving insert, such as a tablet, but designed for rapid dissolution when administered to the rectum or vagina, or when adding to a douche bottle or other douche administration device. In some forms, the drug product is a tenofovir prodrug in a fixed dose combination with another HIV drug (elvitegravir) for rectal and/or vaginal application. In some forms, the drug product is in the form of fast-dissolving powder or granules for dissolution in a suitable buffer without agitation.

### 2. Excipients, Diluents and Fillers

The dosage form may include, or may be reconstituted in an appropriate amount of an aqueous solution, such as water, which can be sterile or non-sterile prior to use. Water includes tap water, spring water, well water, purified or filtered water, distilled water, double-distilled water, deionized water (DI), reverse osmosis-(RO) purified water, RO/DI water, and any other form of water that can safely be used in an enema product and contains insufficient salt to raise the ionic strength of the solution after addition of the powder drug product to physiologically isotonic or hypertonic levels.

A powder dosage form may be reconstituted in an appropriate amount of water, which can be sterile or non-sterile prior to use. Water includes tap water, spring water, well water, purified or filtered water, distilled water, double-distilled water, deionized water (DI), reverse osmosis-(RO) purified water, RO/DI water, and any other form of water that can safely be used in an enema or douche product and contains insufficient salt to raise the ionic strength of the solution after addition of the powder drug product to physiologically hyperosmotic levels. The powder dosage form may be provided in a kit with a suitable container, such as an enema bottle or enema bag, and optionally water.

The compositions include a hypotonic carrier. The hypotonic carrier will typically be a biocompatible liquid carrier that preferably causes little to no signs of irritation when administered to human subjects. The carrier can be naturally occurring or non-naturally occurring including both synthetic and semi-synthetic carriers. Preferred carriers are sodium-based, such as NaOH. Other solutions, including sugar-based (e.g., glucose, mannitol) solutions and various buffers (phosphate-buffers, tris-buffers, HEPES), may also be used.

i. Solutions and Carriers

The TFV formulations for rectal administrations are typically formulated as a hypo-osmolar solution. The terms “hypoosmolar solution”, or “hyposmotic solution” refer to a solution that contains less impermeable solutes compared to the cytoplasm of the cell. A solution with a low concentration of all solutes (including electrolytes, proteins, and nutrients) compared to the concentration in the blood is considered hypoosmolar. Examples of components of hypoosmotic solutions include, but are not limited to, NaOH (for pH adjustment) and NaCl (to adjust the osmolality based upon itself and the other two ingredients); Tris [hydroxylmethyl]-aminomethane hydrochloride (Tris-HCl, 10-100 mM, pH. 6-8), (4-(2-hydroxyethyl)-1-piperazineethanesulfonic acid (HEPES, 10-100 mM, pH 6-8) and dilute solutions of PBS, such as a solution containing 0.2 grams KCl, 0.2 grams KH2PO4, 8 grams NaCl, and 2.16 grams Na₂HPO₄7H₂O in 1,000 ml H₂O, and dilute solutions of normal saline (typically containing 0.9% NaCl).

A hypotonic carrier usually contains water as the major component. The hypotonic carrier can be water, although mixtures of water and a water-miscible organic solvent can also be used. Suitable water-miscible organic solvents include alcohols, such as ethanol, isopropanol; ketones, such as acetone; ethers, such as dioxane and esters such as ethyl acetate. The hypotonic carrier can be water containing one or more tonicity modifying excipients. Sodium chloride is the excipient that is most frequently used to adjust tonicity of a solution. Other excipients used to adjust the tonicity of solutions include glucose, mannitol, glycerol, propylene glycol and sodium sulphate. Tonicity modifying excipients can include pharmaceutically acceptable salts such as sodium chloride, sodium sulfate, or potassium chloride. Other excipients used to adjust tonicity can include glucose, mannitol, glycerol, or propylene glycol.

The tonicity and/or osmolarity of a formulation varies for different cells and mucosal surfaces; it also depends on whether or not the cell or epithelium actively transports solutes and ions; e.g. it has been found that the isotonic point in the vagina for sodium-based solutions is about 300 mOsm/L, similar to the osmolarity of serum, but in the colorectum, it is significantly higher, about 450 mOsm/L (presumable because the colorectum actively transports sodium ions out of the lumen). In some forms the solution has a tonicity from 50 mOsm/L to 280 mOsm/L, from 100 mOsm/L to 280 mOsm/L, from 150 mOsm/L to 250 mOsm/L, from 200 mOsm/L to 250 mOsm/L, from 220 mOsm/L to 250 mOsm/L, from 220 mOsm/L to 260 mOsm/L, from 220 mOsm/L to 270 mOsm/L, or from 220 mOsm/L to 280 mOsm/L, preferably about 145 mOsm.

The hypotonic carrier can include one or more pharmaceutically acceptable acids, one or more pharmaceutically acceptable bases, or salts thereof. Pharmaceutically acceptable acids include hydrobromic, hydrochloric, and sulphuric acids, and organic acids, such as lactic acid, methanesulphonic acids, tartaric acids, and malcic acids. Pharmaceutically acceptable bases include alkali metal (e.g. sodium or potassium) and alkali earth metal (e.g. calcium or magnesium) hydroxides and organic bases such as pharmaceutically acceptable amines. The hypotonic carrier can include pharmaceutically acceptable buffers such as citrate buffers or phosphate buffers.

In some forms, a “wash” douche solution is also provided. Exemplary wash douche solutions include plain water for douching, water with soap, water with salt, or commercial preparations such as saline-based products, mineral oil/glycerin-based products, and laxative-based products.

### 3. Additional Active Agents

In some forms, the hypoosmotic dosage compositions of TFV can contain one or more additional active agents to be delivered including therapeutic agents, prophylactic agents, diagnostic agents, and/or nutraceuticals. “Bioactive agent” and “active agent” are used interchangeably include without limitation physiologically or pharmacologically active substances that act locally or systemically in the body. A biologically active agent is a substance used for the treatment (e.g., therapeutic agent), prevention (e.g., prophylactic agent), diagnosis (e.g., diagnostic agent), cure or mitigation of disease or illness, a substance which affects the structure or function of the body, or pro-drugs, which become biologically active or more active after they have been placed in a predetermined physiological environment. Examples can include, but are not limited to, small-molecule drugs, peptides, proteins, antibodies, sugars, polysaccharides, nucleotides, oligonucleotides, aptamers, siRNA, nucleic acids, and combinations thereof. The agents can be a small molecule (e.g., molecular weight less than 2000, 1500, 1000, 750, or 500 atomic mass units (amu)) or a biomolecule, such as peptides, proteins, nucleic acids, polysaccharides, lipids, glycoproteins, lipoproteins, or combinations thereof. The agents can include one or more of those described in Martindale: The Complete Drug Reference, 37^{th} Ed. (Pharmaceutical Press, London, 2011).

As used herein, the term “Nucleoside and Nucleotide Reverse Transcriptase Inhibitors” or “NRTIs” include those compounds that exhibit anti-HIV effects by inhibiting the activity of HIV reverse transcriptase. In a preferred form, the antiviral is a NRTI in a mildly hypotonic solution. Examples include, but are not limited to, abacavir (ABC), didanosine (ddI), emtricitabine (FTC), lamivudine (3TC), stavudine (d4T), tenofovir (TFV), zidovudine (AZT) and zalcitabine (ddC), and their physiologically functional derivatives. Examples of other classes of antiviral compounds include NNRTIs, protease inhibitors, fusion or entry inhibitors, and integrase inhibitors.

Examples of NNRTIs include, but are not limited to, for example, delavirdine, efavirenz, etravirine, rilpilvirine and nevirapine. Examples of protease inhibitors include, but are not limited to, for example, amprenavir, fosamprenavir, atazanavir, darunavir, indinavir, lopinavir, nelfinavir, ritonavir, saquinavir, and tipranavir. Examples of fusion or entry inhibitors include, but are not limited to, for example, enfuvirtide and maraviroc. Examples of integrase inhibitors include, but are not limited to raltegravir, elvitegravir, and dolutegravir.

Tautomeric forms, isomeric forms including diastereoisomers, and the pharmaceutically-acceptable salts thereof, can also be used. The term “pharmaceutically acceptable salts” embraces salts commonly used to form alkali metal salts and to form addition salts of free acids or free bases. Examples of acids which may be employed to form pharmaceutically acceptable acid addition salts include such inorganic acids as hydrochloric acid, sulfuric acid and phosphoric acid, and such organic acids as maleic acid, succinic acid and citric acid. Other pharmaceutically acceptable salts include salts with alkali metals or alkaline earth metals, such as sodium, potassium, calcium and magnesium, or with organic bases, such as dicyclohexylamine. Suitable pharmaceutically acceptable salts include, for example, acid addition salts which may, for example, be formed by mixing a solution of the compound with a solution of a pharmaceutically acceptable acid, such as hydrochloric acid, sulfuric acid, methanesulphonic acid, fumaric acid, maleic acid, succinic acid, acetic acid, benzoic acid, oxalic acid, citric acid, tartaric acid, carbonic acid or phosphoric acid. All of these salts may be prepared by conventional means by reacting, for example, the appropriate acid or base with the corresponding compounds. Salts formed from free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, and procaine, or other pharmaceutically acceptable salts. Hydrates of the compounds may be used. The term “hydrate” includes but is not limited to hemihydrate, monohydrate, dihydrate, and trihydrate. Hydrates of the compounds may be prepared by contacting the compounds with water under suitable conditions to produce the hydrate of choice.

### 4. Preparation of Formulations

In an exemplary form, a solution of tenofovir is dissolved in water, and is neutralized to pH 7.0±1. In other non-limiting forms, a solution of TFV disoproxil or TFV alafenarnide is dissolved in water to provide a solution with a final pH of 5.2-6, or CMX-157 (potassium form) is dissolved in water to provide solution with a final pH of 7.0±1.

The tenofovir solution may be neutralized with NaOH. Other bases may be used to neutralize the tenofovir, such as KOH or Mg(OH)₂. A sodium phosphate buffer also may be used to neutralize the tenofovir, followed, optionally, by grinding or dry granulation with sodium phosphate (rather than NaCl, as described below).

In an exemplary form, a neutralized solution is lyophilized or spray-dried, as is common in the pharmaceutical industry. The particles may be comminuted if lyophilized to produce a more uniform particle size distribution. Tenofovir+NaOH particles are quite hygroscopic and do not flow well. As such, after drying the particles are maintained in a low humidity environment of air, an inert gas such as Ar, or nitrogen gas, having a low relative humidity, for example a relative humidity of less than 35%, 30%, 25%, or 20%, such as ranging from 20% to 25%, including any integer or increment therebetween. The dried tenofovir is then ground with a salt, such as NaCl, monosodium phosphate, disodium phosphate, potassium monobasic phosphate, potassium dibasic phosphate, potassium chloride, magnesium chloride, or a mixture of any of the preceding, such as a mixture of monosodium phosphate and disodium phosphate, to produce flowable particles that include intermingled tenofovir and salt. In non-limiting forms, another excipient, such as a sugar, may be added to improve flowability. The particles may be then packaged, e.g., in a sachet or other container. In non-limiting forms, tenofovir, at a concentration of 110 mg/mL (e.g., 11% w/w), may be prepared with NaOH, and spray drying may be performed to provide a powder, where spray drying may be performed with the inlet set at about 200 °C, pump flow set at about 4 mL/min, aspirator set at about 35 m3/h, and N2 flow set at about 601 L/h. When dried and combined with sodium chloride crystals, a powder with suitable followability may be achieved. For example, in some forms, the spray drying includes an inlet temperature of about 200 °C, a pump flow rate of about 4 mL/min, an aspiration rate of about 35 m3/h, and an N2 flow rate of about 601 L/h.

### 5. Exemplary Formulations

The most commonly used commercial douche formulations rely on their hypertonicity to cause a large and rapid influx of water to cleanse the rectal vault. However, hypertonic sexual lubricant gels cause significant loss of single columnar epithelium within minutes of applying a single dose. In contrast, when an “isoosmolar” (osmolarity similar to that of blood) lubricant was used, there was no loss of single columnar epithelium. Other studies have also reported mucosal damage due to hypertonic solutions. A large proportion of users simply use tap water (a strongly hypotonic formulation) due to easy availability, however, some studies suggest epithelial loss (toxicity) after its use. To address the potential use of douche formulations of tenofovir or a derivative thereof, either alone or as a complement to another method, experiments were designed to assess the safety, distal gastrointestinal distribution, retention, and acceptability (as set forth in the Examples). In exemplary forms, the TFV is in an amount of about 100 ml-150 ml, such as about 125 ml.

As used herein, the term “mildly hypoosmotic” means an aqueous solution or carrier having an osmolarity greater than tap water (0 mOsm), and between about 20 mOsm to about 290 mOsm. In some forms, the hypoosmotic formulation of tenofovir or a derivative thereof have osmolarity in the range of 200 mOsm to about 240 mOsm, preferably about 220 mOsm. The tenofovir or a derivative thereof are preferably formulated using known buffers and salts, including, for example, normal saline, phosphate buffered saline (PBS), Tris-buffered saline (TBS), or HEPES buffered saline.

As demonstrated by the examples, rapid absorption of TFV into plasma, was attributable to the advective forces of the hypo-osmolar fluid moving solute molecules rapidly to the colorectal mucosal surface in the first moments after dosing prior to establishing physiological iso-osmolar concentrations in the colorectal lumen. Following this, a second peak, was consistent with a slower and sustained absorption from residual drug in the colorectal lumen.

As set forth in the Examples, a douche formulation including a hypoosmotic solution of 660 mg TFV that has an osmolality of approximately 145 mOsm/Kg gave the best results. The solution contained both 0.33% NaCl pH 7.0=1.0, adjusted with sodium hydroxide (18% w/v) and 0.1M HCL. Each application contained 660 mg TFV in 125 mL, and it was found that fluid absorption occurred more rapidly due to a hypo-osmolar solution administered to the rectal lumen. In some forms, the final NaCl concentration is about 0.45% (e.g., about “half” normal saline) Therefore, in some forms, the formulation includes an amount of approximately 1,320 mg of TFV, as well as NaCl, NaOH, and, optionally if needed to adjust pH, HCl and/or NaOH, where the dose of TFV, when reconstituted, is 5.28 mg/mL in a volume of approximately 250 ml. In some forms, the dosage form is packaged suitable for intrarectal administration as two separate amounts of 125 ml each. It has been determined that one 660 mg tenofovir dose in 125 mL administered only once achieves a target TFV concentration in the rectal lumen, and a series of 3 sequential douches of 660 mg in 125 mL (total dose of 3×660 mg and total volume administered 3×125 mL) achieves the same concentrations. However, a single tenofovir douche (660 mg in 125 mL) followed by tap water douches, but no ending tenofovir douche results in lower concentrations. Net result is not quite as high peak and shorter duration; a single tenofovir douche (660 mg in 125 mL) preceded by only tap water douches also results in lower concentrations than a single or a series of 3 tenofovir douches. Accordingly, the combination of the initial douche being medicated with tenofovir and the final douche being medicated with tenofovir will restore much of the loss associated with a non-medicated wash with only the first or only the last douche containing tenofovir.

Therefore, in some forms, unit dose sachets of TFV douche contain TFV 660 mg. Upon reconstitution in 125 mL tap water, as directed, this provides a final concentration of 5.28 mg/mL TFV in hypo-osmolar (145 mOsm/kg) solution. The hypo-osmolar characteristic of the solution is due to final concentration of 0.45% NaCl.

### 6. Packaging, Containers and Vessels

The dosage form, such as a powder, may be provided in a kit with a suitable container, such as an enema bottle or enema bag, and optionally water. Suitable containers for delivery include, without limitation, an enema bag, an enema bottle, an enema bottle having an extended tip, medical syringes or droppers, containing a composition including an active ingredient useful for PrEP.

Therapeutic compositions may be sterile, and should be stable under the conditions of manufacture and storage. For example, sterile powders can be prepared by incorporating the active agent in an appropriate solvent with one, or a combination of ingredients, followed by filter-sterilization. The liquid may be spray-dried, vacuum dried, lyophilized, or otherwise dried, under aseptic conditions, and packaging, such as sachets or other containers, may be sterilized prior to filling with the powder described herein under aseptic conditions. Once filled, the sachet or other container may be further sterilized, e.g., in a manner that does not, or does not substantially deteriorate the powdered drug product within the sachet or other container.

Stick packs and sachets are common packaging products in many industries, including the food and pharmaceutical industries. They typically include, or are formed from, a polymeric membrane, such as MYLAR or a MYLAR foil laminate. Stick pack pouches can include an elongated tube-shaped, e.g., cylindrical pouch with a fold-over side and an optional tear notch, which can be formed from tubular materials that are sealed at both ends. The tube may have a continuous side seam. A stick pack typically has a length at least three or at least four times the width or diameter of the tube. Stick packs may include a tear notch, arranged at any suitable location to allow for a user to easily open the pack. Sachets are typically flat or bulging pouches sealed at three or four sides, often including a tear notch, which may be arranged at any suitable location to allow for a user to easily open the sachet. The tear notch may be adjacent to a spout portion of the sachet extending into the seal and configured to provide an opening narrower than a dimension of the sachet.

In some forms, a dosage form of TFV is configured for application using a rectal delivery device. A rectal delivery device is a device used to deliver the formulations or compositions described herein to the rectum. Traditional rectal delivery devices have been used for localized treatments including delivery of laxatives, treatment of hemorrhoids, and for delivery of antipyretics. Rectal delivery devices include, but are not limited to, an enema bag or an enema bottle, e.g., having an extended tip.

Exemplary devices used for RD include enema bottles, rubber bulbs, attachable hoses, commercial douche kits, and plastic tubing.

### D. Kits

Kits of parts for use according to the described dosing regimens are also provided.

Typically, the kits include at least a dosage form of tenofovir or a derivative thereof, including a prophylactically effective amount to inhibit HIV infection in a subject for a period of at least 24 hours, up to 96 hours or 168 hours, following rectal administration, together with one or more additional components, reagents or packaging inserts. In some forms, the kits include the dosage form within one or two hermetically sealed container(s). In some forms, the dosage form of the kit includes 9-[9 (R)-2-(phosphonomethox y) propyl] adenine (PMPA). In some forms, the dosage form of the kit includes a tenofovir derivative such as tenofovir disoproxil fumarate (TDF), tenofovir exalidex and tenofovir alafenamide (TAF). In some forms, the dosage form of the kit includes tenofovir or a derivative thereof in an amount of between about 1,000 mg and about 1,500 mg, inclusive, such as about 1,320 mg. In some forms, the dosage form of the kit includes one or more of NaCl, NaOH, or NaHCO₃. In some forms, the tenofovir or derivative thereof is in the form of a solid, a liquid, or a semi-solid, such as a solid selected from a powder, a tablet, and lyophilized or dried material. An exemplary powder includes particles of between about 20 μm to about 600 μm in diameter, inclusive in an amount to yield, when reconstituted as an aqueous solution, a hypotonic solution of from 145 mOsm/kg to about 290 mOsm/kg including from 0.1 mg/ml to 20 mg/ml, 1.8 mg/ml to 10 mg/ml, 2 mg/ml to 10 mg/ml, or 5.28 mg/ml, of tenofovir or a therapeutic or molar equivalent amount of a derivative thereof.

In some forms, the kit includes a container, such as a cylindrical container including a polyester, such as a biaxially oriented polyethylene terephthalate, or a biaxially-oriented polyethylene terephthalate that is metallized.

In some forms, the dosage form of the kit includes a sterile, lyophilized powder of TFV in an amount of about 1,320 mg, wherein the powder is packaged within a hermetically sealed container, wherein the hermetically sealed container includes two compartments, and wherein each compartment includes approximately 660 mg of the powder. In some forms, the kit further includes instructions according to a dosing regimen described herein. For example, in some forms, the kit includes instructions for the administration of the dosage form of TFV by administering into the rectum of a subject an effective amount of tenofovir or a derivative thereof to inhibit infection or replication of HIV in the rectum and/or intestine of the subject for a period of at least 24 hours, up to 96 hours, or up to 168 hours following administration, including administering the tenofovir or a derivative thereof into the rectum in the form of an aqueous solution, wherein the aqueous solution is applied into the rectum as a first and a second discrete applications, wherein each of the first and second discrete applications into the rectum are separated in time by less than 60 minutes.

In some forms, the kit further includes an aqueous solution.

The present invention will be further understood by reference to the following non-limiting examples.

### EXAMPLES

### Example 1: Single Douche Dose and Osmolality Comparison DREAM-01

To establish suitable methods and compositions for a Tenofovir douche a Phase 1 Trial, including a single ascending dose study of 3 formulations of a TFV rectal douche was performed in healthy MSM without HIV to identify the dose and osmolarity that achieves target colonic tissue cell TFV-DP concentrations that exceed the Pre-exposure Prophylaxis Initiative (iPrEX) tissue EC90.

### Method

Escalating doses were: Product ATFV 220 mg in125 mL normal saline, Product B 660 mg TFV in 125 mL normal saline, and Product C 660 mg TFV in 125 mL half-normal saline. TFV PK was assessed through semi-intensive sampling of plasma and PBMC and sparse sampling of rectal fluid and colon tissue was performed. Rectosigmoid product distribution was assessed in 6 participants using 99Tc-DTPA labeled douche and quantitative SPECT/CT imaging. PD were assessed using ex vivo rectal tissue explant HIV challenge. Acceptability was assessed via questionnaires and in-depth interviews.

### Results

The douche was safe and well tolerated, with high acceptability to participants (Table 1).

TABLE 1
Safety and Acceptability of Formulations

| | | | | | | Serious |
|---|---|---|---|---|---|---|
| Study | N | Grade 1 | Grade 2 | Grade 3 | Attrib. | AE |
| DREAM-01 | 21 | 56 | 2 | 3 | 2 | 1 |
| DREAM-02 | 9 | 5 | 6 | 0 | 1 | 0 |
| DREAM-03 | 6 | 27 | 12 | 0 | 2 | 0 |
| Total | 36 | 88 | 20 | 3 | 5 | 1 |

Adverse events (AE) reported in DREAM-01, DREAM-02, and DREAM-03 by DAIDS grading, including attribution to study products and procedures and serious AEs. All attributable events were Grade 1, reversible, and included case(s) of pelvic discomfort (2), rectal dryness (1), bloating (1), and blood-tinged mucus (in a participant with history of inflammatory bowel disease).

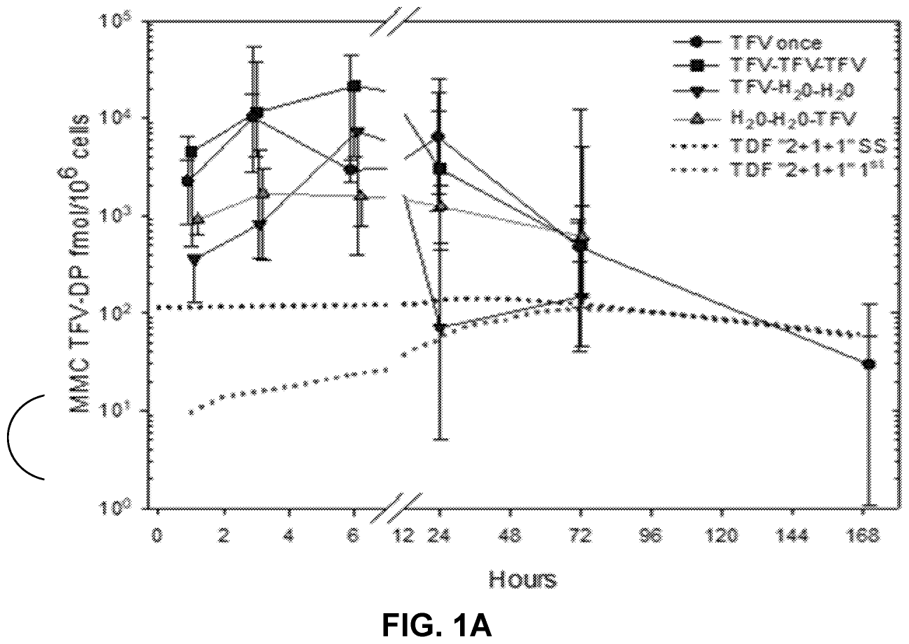

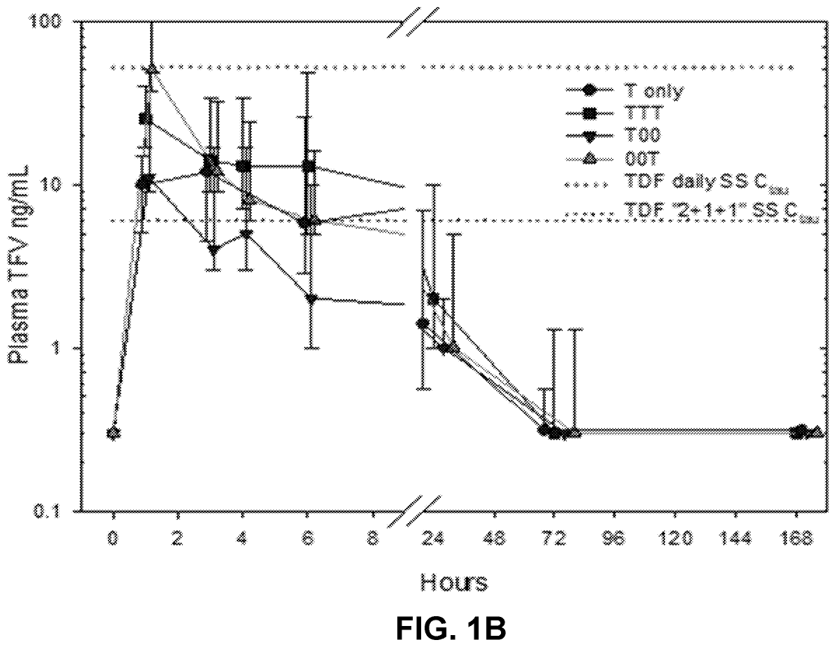

Peak plasma TFV concentrations (Cmax) were 4-fold lower than trough (Cmin) concentrations associated with steady-state oral TDF daily dosing. Within 1-3 hours after dosing, colon tissue cell active TFV-DP concentrations exceeded steady-state oral on-demand 2-1-1 dosing regimen rectal tissue cell TFV-DP target concentrations from 1.4 log 10 (Product A) to 2.0 log 10 (Products B and C); rectal tissue cell TFV-DP concentrations following a single dose of the hypo-osmolar, high dose Product C remain above model-based estimates achieved with the oral 2-1-1 F/TDF regimen for 5 or 6 days (FIGS. 1A-1B). Pharmacodynamic assessment using ex vivo colon tissue explants HIV challenge indicated median (interquartile range [IQR]) colon tissue cell TFV-DP IC50 2,192 fmol/106 cells (595, 3,789) and Imax 1.9 log 10 pg/mL/mg (1.6, 2.3). This is the first study of any PrEP product indicating a formal PK/PD-defined concentration response using the ex vivo explant HIV challenge assay. In MTN-017—which included oral daily F/TDF tablets, rectal daily TFV RGVF gel, and rectal on-demand (RAI-associated) TFV RGVF gel—none of the study arms achieved full suppression of HIV replication in explants, and concentrations were typically well below DREAM-01 concentrations on the day of dosing, as shown in FIG. 2.

### Example 2: Triple Douche Sequence Comparison (DREAM-03)

To determine the optimal triple douche sequence comparison, a Phase 1, open label study compared the safety, PK, PD, and acceptability of three sequences of the Product C TFV douche (selected for advancement from DREAM-01) and non-medicated tap water douches.

### Methods

DREAM-03 investigators selected three douche sequences based upon an international (US, Latin America, and Sub-Saharan Africa) sexual behavior survey of MSM/TGW, which reported that, among those who douche prior to RAI, three douches in rapid succession are typically used to achieve satisfactory cleansing, over a range of one to five douches. DREAM-03 investigators hypothesized that three TFV-medicated douches, in comparison to sequences that also contained tap water douches, would achieve the highest colon tissue TFV-DP concentrations, though these concentrations were expected to be similar to a single TFV douche, with the least dosing complexity, but at greatest cost of goods. A douche sequence that either began or ended with two tap water douches, tap water being the most common type used based on surveys, before or after a single medicated TFV douche, would be more complicated and result in lower tissue TFV-DP concentrations, but less expensive. The DREAM-03 dosing and multiple matrix sampling scheme largely mimicked that of DREAM-01 in nine MSM without HIV.

### Results

DREAM-03 demonstrated a similar adverse event profile as DREAM-01 (Table 1) as well as no changes in histology compared to baseline (Table 2).

TABLE 2
ADVERSE EVENT PROFILE AND HISTOLOGY

| | | | |
|---|---|---|---|
| | IBD Scale | Epithelial | Lamina Propria |
| Study | (Scale 0-5) | Denudation | Hemorrhage |
| Test | Median | (Scale 0-3) | (Scale 0-3) |
| Condition | (IQR) | Median (IQR) | Median (IQR) |

DREAM-01 Single
Douche with Dose Escalation and Osmolality De-escalation

| | | | |
|---|---|---|---|
| Baseline | 0 (0, 1) | 0 (0, 1) | 1 (0, 1) |
| TFV 220 mg, | 0 (0, 1) | 0 (0, 1) | 1 (0, 1) |
| NaCl 0.9% | | | |
| TFV 660 mg, | 1 (0, 1) | 0 (0, 0.8) | 1 (0.2, 1) |
| NaCl 0.9% | | | |
| TFV 660 mg, | 0 (0, 1) | 0 (0, 1) | 1 (1, 1) |
| NaCl 0.45%* | | | |

DREAM-03 Three Douche Sequence with Douche Type Variation*

| | | | |
|---|---|---|---|
| Baseline | 1 (0, 1) | 0 (0, 0) | 0 (0, 0.5) |
| TFV - TFV - TFV | 1 (0.8, 1) | 0 (0, 1) | 0 (0, 1) |
| TFV - H₂O - H₂O | 1 (1, 1) | 0 (0, 0.2) | 0 (0, 1) |
| H₂O - H₂O - TFV | 1 (0.8, 1) | 0 (0, 0) | 0 (0, 1) |

PK analysis confirmed investigator hypotheses that a sequence of three hypo-osmolar TFV douches in rapid succession (Product C, 660 mg TFV in 125 mL 0.45% NaCl) achieved roughly similar colorectal tissue TFV-DP concentrations in cells extracted from tissue biopsies when compared to a single dose of the same formulation in DREAM-01, though plasma TFV concentrations are moderately higher with the triple dose compared to a single dose (FIGS. 1A-1B).

Tissue cell TFV-DP concentrations with the douche sequences containing two tap water douches and one TFV douche were both lower than the single or triple TFV douche sequence. Plasma TFV PK following the douche sequence ending with two tap water douches fell below all other single (DREAM-01) and triple douche (DREAM-03) sequences containing the Product C douche. The sequence that ended with the Product C douche had the highest plasma TFV concentrations. Regarding comparison with oral TDF dosing, all three douche sequences resulted in plasma TFV trough (Ctau) concentrations above those typically associated with the 2-1-1 regimen, but below typical Ctau for a daily regimen. Taken together, these findings suggest a complicated pattern of colorectal tissue cell and plasma drug uptake driven largely by luminal colorectal TFV concentration during the first 24 hours after dosing.

The douche regimen selected for the HPTN 106 protocol is derived from PK data from DREAM-01 and DREAM-03 together in light of the heterogeneity of douching practices from a Grindr survey. Beyond these formal sources, community feedback indicated substantial heterogeneity in individual douche number (or volume for those who use hoses). Guided by these studies and community feedback, a “bookends” TFV douche regimen was selected for the study that begins with the TFV douche, followed by the usual douche practices (including delivery method, number, and volume of douching), followed finally by a terminal TFV douche. This provides the advantages of rapid uptake and high tissue cell active TFV-DP concentration following the leading TFV douche and the advantage of leaving the colonic lumen with study product and its related increased duration of tissue cell TFV-DP throughout the week after dosing. Modeling (based on DREAM-01 and DREAM-03) and simulation of this TFV douche bookends approach (TFV douche, typical douching, final TFV douche) indicates only modest reductions in peak tissue cell TFV-DP compared to only douching with the TFV douche product.

### Example 3: Anal Sex and Tenofovir Douche Sequence Impacts Colorectal Distribution of HIV Surrogate and Douche (DREAM-02)

DREAM-02 assessed the safety and colonic luminal distribution of a Product C douche either preceded or followed by simulated RAI (sRAI) to understand the impact of timing of sex relative to TFV douching on colon distribution. To understand the luminal interactions of RAI and a medicated rectal douche, SPECT/CT was used in a phase I study to assess intraluminal colonic distribution of both ^{99m} technetium (^{99m} Tc)-labeled HIV surrogate, and ¹¹¹indium (¹¹¹In)-labeled TFV douche, where TFV douche was rectally administered either preceding or following sRAI. Clinical safety of the study product/procedure and mucosal permeability of radiotracers and TFV were also assessed.

### Methods

A phase 1, open-label study in HIV-negative individuals was performed to assess if varying sequences of a TFV douche and sRAI would affect the distribution of douche solution and an HIV surrogate in seminal fluid. Study endpoints included clinical safety, intraluminal colonic distribution of the TFV douche and autologous seminal fluid, and systemic exposure of radiotracers and TFV. The study was conducted in the Johns Hopkins Drug Development Unit. The study protocol (IRB #00208338) was approved by the Johns Hopkins University Institutional Review Boards and registered in ClinicalTrials.gov

HIV-negative adults (18 years of age or older) that were biologically assigned male at birth were recruited, provided informed consent, and screened at the Johns Hopkins University (Baltimore, MD). Inclusion criteria included a history of consensual RAI and experiences with receiving or self-administering a rectal douche in the context of RAI. Exclusion criteria included specified abnormalities in laboratory chemistries, history of chronic Hepatitis B infection, active rectal or reproductive tract infection, significant colorectal symptoms, and whole-body radiation exposure exceeded 5000 mrem/year.

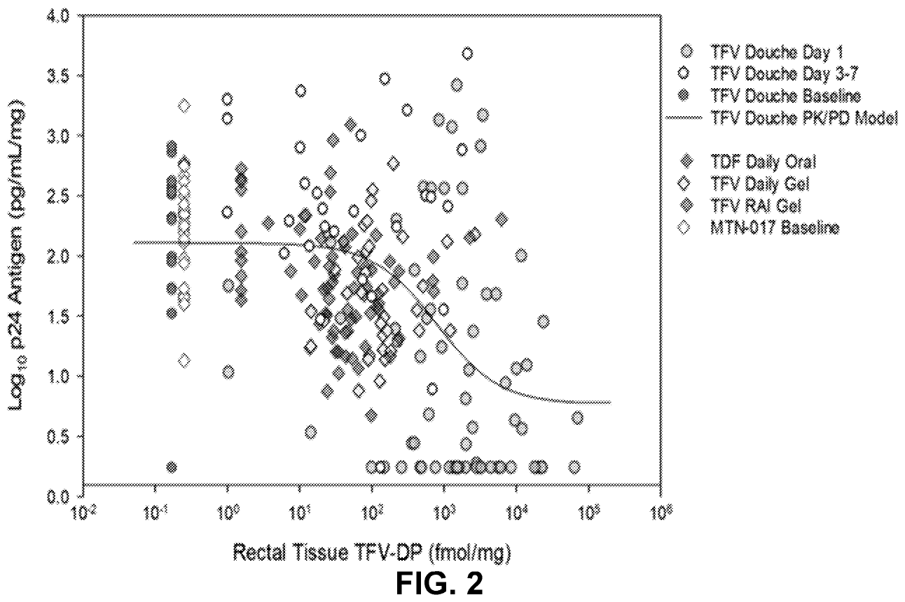

After enrollment, participants underwent protocol-defined study visits preparing for each product sequence, administration of both douche and sRAI sequences, SPECT/CT imaging and sample collection following each sequence, and safety assessment, as set forth in the schedule depicted in FIG. 3.

At Sequence A visit, the radiolabeled TFV (660 mg) douche was administered rectally. The douche included of 0.33% NaCl (osmolality 145±22 mOsm/kg), pH 7.0±1.0, adjusted with sodium hydroxide (18% w/v) and 0.1M HCL. It contained 660 mg TFV in 125 mL, mixed with 0.5 mL of 200 μCi/mL ¹¹¹indium-diethylenetriaminepentaacetic acid (¹¹¹In-DTPA) as a small molecule surrogate in solution.

The douche was retained less than 1 minutes, then expelled. The douche was followed by sRAI for 5 minutes using insertion and manipulation of an artificial phallus to simulate RAI. Ejaculation was simulated via injection of a semen-HIV surrogate mixture through a catheter in the urethral position of the phallus. The semen-HIV surrogate mixture was prepared with 2.5 mL autologous seminal fluid and 0.5 mL of 1 mCi/mL ^{99m} technetium-sulfur colloid (^{99m} Tc-SC), a cell-free HIV surrogate 100 nm particle in colloidal suspension. Blood samples were collected over 60 minutes post-dose. SPECT/CT images were acquired at 60 minutes after the sRAI process.

After a washout period of at least 14 days, at the Sequence B visit, sRAI with autologous seminal fluid was first performed, followed by douche administration. Autologous seminal fluid was collected during Pre-A and Pre-B visits for use in sRAI.

Safety of study procedures and products was closely monitored for the entire duration of this study. At screening, Sequence A, Sequence B, and the final visit, participants were interviewed to report interval medical history and adverse events. General physical examinations to assess vital signs and safety laboratory examinations including sexual transmitted infection testing, complete blood count with differential, comprehensive metabolic panel, and quantification of serum phosphate levels were also performed at each visit. In addition, participants were instructed to contact study site staff to report any adverse events that they experienced at any time between enrollment and completion of this study.

### Data Collection and Analysis

Luminal distribution of HIV surrogate (^{99m} Tc-SC) and TFV douche surrogate (¹¹¹In-DTPA) were determined using SPECT/CT on a dual-head VG SPECT series system (GE Medical Systems, Waukesha, WI) equipped with a CT unit (Hawkeye). Images were processed and reconstructed into a 256×256×256 matrix, with a voxel size of (1.72 mm)³, using a General Electric eNTEGRA workstation with software version 1.04 (GE Medical Systems, Waukesha, WI) as previously described. Curve-fitting to estimate colonic distribution distances was performed using reported algorithms with R Studio version 4.2.2 (The R Foundation for Statistical Computing, Vienna, Austria). The penalty coefficient, lambda 1, was set to 1000 for tube-shape signals and 4000 for blob-shape signals to reduce overfitting. The anorectal junction (ARJ) was used as the reference origin (set to 0 cm with proximal distribution “+” values and distal distribution “−” values).

Colorectal pharmacokinetic-distance parameters, including Dₘᵢₙ (distance associated with most distal radiolabel signal within the colon), Dₘₐₓ (distance associated with most proximal radiolabel signal within the colon), Dₛₚₐₙ (calculated as Dₘₐₓ−Dₘᵢₙ to assess the span of colonic distribution of radiolabeled signal), DCₘₐₓ (distance associated with the maximum radiotracer concentration), Dₐᵥₑ (mean residence distance), and Cₘₐₓ (signal intensity within volume equivalents of a theoretical plate moved along the central line of signal) were calculated.

Amounts of ^{99m} Tc-SC and 111 In-DTPA were quantified in 1 mL aliquots of collected plasma samples on a gamma counter (Wizard2 automatic gamma counter model 2480, PerkinElmer, Waltham, MA) using a 110- to 150-keV energy window, and the results were corrected for decay relative to the time of TFV douche administration. Plasma TFV concentrations were quantified by the Johns Hopkins Clinical Pharmacology Analytical Laboratory using validated liquid chromatography-tandem mass spectrometr (UPLC-MS/MS) methods. The linear range of analytical quantification was 1 to 1000 ng/mL. For participants who had quantifiable baseline plasma TFV, post-dose TFV concentrations were corrected by subtracting estimated TFV concentrations, assuming baseline TFV would be eliminated following a 17-hour half-life. Non-compartmental analysis was performed to obtain PK parameters, including time to peak concentration (Tₘₐₓ), peak concentration (Cₘₐₓ), half-life (T_{1/2}), and area under curve over the sampling period (AUCₗₐₛₜ) using the Phoenix WinNonlin version 8.2.0 (Certara, Wilmington, DE).

Statistical analysis was performed using STATA SE version 17.0 (StataCorp LP, College Station, TX). Wilcoxon signed-rank tests were performed to compare the data collected during Sequences A and B. A P value≤0.05 was considered statistically significant.

### Results

### Study Participants

Six participants were consented, screened, and enrolled in this study. Five participants completed paired study Sequences A and B and were included for analysis (see FIG. 3). These participants were all white cisgender men with a median (range) age 31 (25 to 40) years, weight 86 (62 to 95) kilograms, and body mass index (BMI) 24.9 (22.8 to 28.4).

### Adverse Events

There were five Grade 1 adverse events, including hypophosphatemia (one event during Sequence A visit and one during final follow-up visit), pelvic discomfort (one occurrence during staff administrations of the TFV douche during the Sequence A visit), tenesmus/incomplete evacuation (one event not during a visit), and non-fasting hyperglycemia (one event during Sequence B visit). Only one event (pelvic discomfort) was deemed related to the study procedure. This event occurred during douche administration and was resolved within the same day.

### Colonic Distribution of TFV Douche and Seminal Fluid

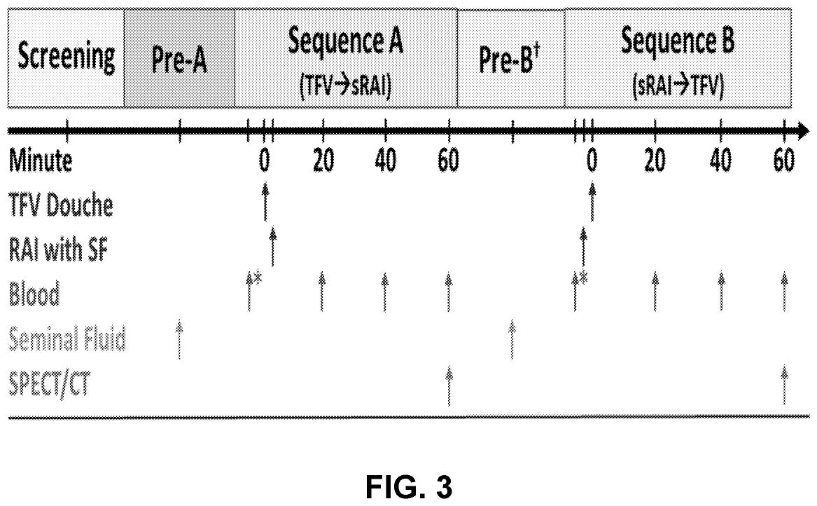

In visual comparisons of douche and HIV surrogate distributions, three out of five participants in Sequence A showed greater distribution of TFV douche than seminal fluid in the colorectum, while the other two participants exhibited similar distribution of TFV douche and seminal fluid. In Sequence B, four out of five participants had similar distributions of TFV douche and seminal fluid, while in one participant, the distribution of seminal fluid was relatively greater than that of TFV douche. All five participants had greater distribution of HIV surrogate (Dₛₚₐₙ) in sequence B when compared to Sequence A (FIG. 4, Table 3). The luminal distribution of radiolabel was quite variable for both douche surrogate (Dₛₚₐₙ ranged from 6.3 cm to 43.5 cm in Sequence A and 10.6 cm to 36.4 cm in Sequence B) and HIV surrogate (Dₛₚₐₙ ranged from 7.1 cm to 13.5 cm in Sequence A and 12.1 cm to 39.6 cm in Sequence B). Comparing sequences, Sequence B trended higher for both Dₘₐₓ (2.5-fold), Dₛₚₐₙ (3.2-fold), and Dₐᵥₑ (1.8-fold) of the HIV surrogate when performing paired comparison to Sequence A (all p=0.06). The Douche surrogate was not different between sequences. Results of quantitative analysis were consistent with visual impression.

TABLE 3
Estimates of SPECT/CT Pharmacokinetic-
Distance parameters, median (range).

| | Sequence A | Sequence B | p-value |
|---|---|---|---|
| Dₘᵢₙ (cm) | | | |
| ¹¹¹In-DTPA | 5.4 (2.9, 6.9) | 3.7 (2.9, 8.4) | 0.81 |
| ^{99m}Tc-SC | 3.6 (2.4, 6.3) | 2.2 (1.5, 7.4) | 0.31 |
| Dₘₐₓ (cm) | | | |
| ¹¹¹In-DTPA | 16.3 (11.7, 50.4) | 32.5 (14.3, 43.0) | 0.44 |
| ^{99m}Tc-SC | 13.4 (12.1, 17.1) | 33.7 (14.3, 47.0) | 0.06 |
| Dₛₚₐₙ (cm) | | | |
| ¹¹¹In-DTPA | 11.8 (6.3, 43.5) | 27.9 (10.6, 36.4) | 0.44 |
| ^{99m}Tc-SC | 9.8 (7.1, 13.5) | 31.5 (12.1, 39.6) | 0.06 |
| Dₐᵥₑ (cm) | | | |
| ¹¹¹In-DTPA | 9.5 (7.6, 24.1)^{†} | 14.5 (8.1, 24.1) | 0.31 |
| ^{99m}Tc-SC | 7.7 (6.7, 9.5) | 14.0 (7.5, 28.6) | 0.06 |
| DCₘₐₓ (cm) | | | |
| ¹¹¹In-DTPA | 7.5 (6.0, 18.1) | 8.8 (4.8, 23.3) | 0.81 |
| ^{99m}Tc-SC | 8.2 (6.6, 9.2) | 7.2 (5.4, 40.2) | 0.31 |
| Cₘₐₓ | | | |
| (I/VEq) | | | |
| ¹¹¹In-DTPA | 3.4 (2.1, 7.6)^{†} | 4.1 (2.6, 5.6)^{†} | 1.00 |
| ^{99m}Tc-SC | 45.4 (39.2, 54.7) | 9.5 (8.1, 12.5) | 0.06 |

I/VEq: intensity per volume equivalents;
^{†}p = 0.06 when compared to ^{99m}TC distributional parameters in the same sequence

### Systemic Exposure of TFV and Radiotracers

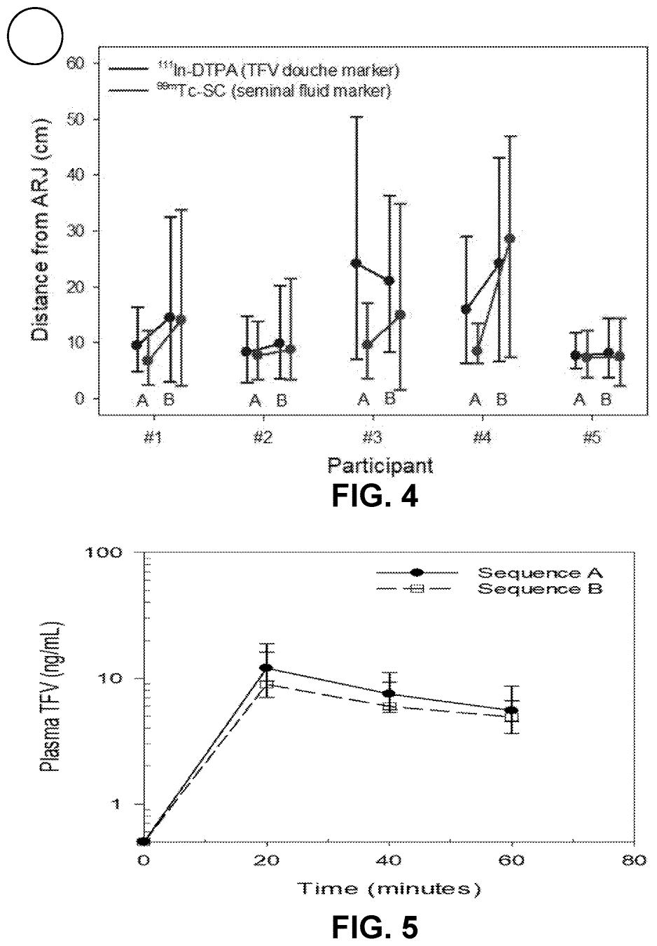

Plasma TFV concentrations were quantifiable in all participants. However, one participant took a dose of oral TFV alafenamide/emtricitabine (F/TAF) on the same day of Sequence A visit and one participant took a dose of oral TFV disoproxil fumarate/emtricitabine (F/TDF) on the same day of Sequence B visit, which substantially interfered with PK analysis due to the much higher plasma TFV concentrations soon after oral dosing when compared to rectal dosing. Therefore, results from these two visits were excluded from plasma TFV PK analysis. In both administration sequences, the highest observed concentrations during the first hour after douching occurred 20 minutes post-dose (FIG. 5). Plasma TFV concentrations were similar between sequences at all time points with similar rate of decay; zero-to-one hour PK parameters, including Cₘₐₓ, Tₘₐₓ, T_{1/2}, and AUC₀₋ₗₐₛₜ, were similar between sequences (Table 4). These findings suggested that the sequence of douching and sRAI had minimal impact on systemic TFV exposure within the first hour post-dose.

TABLE 4
Plasma TFV concentrations rate of decay; zero-to-one hour
PK parameters, including Cₘₐₓ, Tₘₐₓ, T_{1/2}, and AUC₀₋ₗₐₛₜ

| | Sequence A | Sequence B |
|---|---|---|
| Tₘₐₓ (hr) | 0.33 (0.33, 0.33) | 0.33 (0.33, 0.33) |
| Cₘₐₓ (ng/mL) | 12.00 (8.98, 17.13) | 8.94 (6.74, 21.9) |
| C₆₀ | 5.54 (4.25, 6.93) | 4.91 (3.38, 9.75) |
| T_{1/2} (hr) | 0.70 (0.43, 1.46) | 0.70 (0.44, 2.22) |
| AUCₗₐₛₜ (ng · hr/mL) | 7.91 (5.71, 8.43) | 5.58 (4.89, 13.02) |

Table 4: Estimates of TFV 8 PK parameters, median (range). For all parameters, p=1.000; Tₘₐₓ: time to peak concentration; Cₘₐₓ: peak concentration; C60: concentration at 60 minutes; T_{1/2}: decay half-life; reported estimated distributional decay T_{1/2}, d=0.71 hrs; AUCₗₐₛₜ: area under curve over the sampling period.

Plasma ^{99m} Tc-SC concentrations were less than 0.00006% of the dosed ^{99m} Tc-SC concentration in two participants and below detection limits in the other three participants in both sequences. There was no quantifiable plasma ¹¹¹In-DTPA in any participant.

### Discussion

Preliminary descriptive results of SPECT/CT imaging indicate a profound effect of the order of TFV douche and sRAI sequence (Table 5).

TABLE 5
SPECT/CT RESULTS ACCORDING TO SEQUENCE OF MICROBICIDE DOSING AND RAI.

| | | [symbol] In TFV | [symbol] TC [symbol] HIV [symbol] | |
|---|---|---|---|---|
| PID | Sequence | Douche | Surrogate | Distribution |
| J202 | TFV douche [symbol] > sRAI | R to S | R | TFV > [symbol] HIV [symbol] |
| J202 | sRAI [symbol] > TFV douche | R to DC | R to DC | TFV = [symbol] HIV [symbol] ; increased distribution v. douche [symbol] > sRAI |
| J203 | TFV douche [symbol] > sRAI | R to S | R to S | TFV = [symbol] HIV [symbol] |
| J203 | sRAI [symbol] > TFV douche | R to DC | R to DC | TFV = [symbol] HIV [symbol] ; increased distribution v. douche [symbol] > sRAI |
| J204 | TFV douche [symbol] > sRAI | R to DC | R | TFV > [symbol] HIV [symbol] |
| J204 | sRAI [symbol] > TFV douche | S to DC | R to DC | [symbol] HIV [symbol] > TFV; increased distal distribution v. douche [symbol] > sRAI |
| J205 | TFV douche [symbol] > sRAI | R to DC | R | TFV > [symbol] HIV [symbol] |
| J205 | sRAI [symbol] > TFV douche | R to SF | R to SF | TFV = [symbol] HIV [symbol] ; increased distribution v. douche [symbol] > sRAI |
| J206 | TFV douche [symbol] > sRAI | R | R | TFV = [symbol] HIV [symbol] |
| J206 | sRAI [symbol] > TFV douche | R to S | R to S | TFV = [symbol] HIV [symbol] ; increased distribution v. douche [symbol] > sRAI |

R: Rectum;
S: Sigmoid colon;
DC: Descending Colon;
SF: Splenic Flexure [symbol]
indicates data missing or illegible when filed

Douche prior to sRAI shows a consistent pattern of greater douche distribution within the colon with overlap of HIV surrogate distribution largely confined to the rectum, sometimes to the sigmoid colon. When sRAI precedes the TFV douche, the distribution of both douche ^{99m}Tc-DTPA and 111 In-sulfur colloid HIV surrogate are similar, which indicates a much greater colonic distribution of the HIV surrogate. The implications of the redistribution of the HIV surrogate with regard to HIV acquisition are uncertain and complex as there is both a larger colonic distribution of the HIV surrogate as well as a substantial dilution of the HIV surrogate as well. In silico modeling of viral dynamics may be useful to better understand these, among other, variable effects on HIV infectivity. With regard to the TFV douche, the distribution is largely reproducible within an individual, regardless of sequencing related to RAI and there is a trivial dilutional effect by the small 2-3 mL ejaculate volume compared to the much larger 125 mL TFV douche volume with each unit dose used.

Intraluminal colonic distribution of the TFV douche was similar whether preceding or following sRAI, but the distribution of the HIV cell-free surrogate delivered during sRAI was greater (more proximal for Dₘₐₓ and Dₐᵥₑ), often extending into the descending colon, when sRAI preceded the TFV douche rather than following it.

When sRAI followed the TFV douche, the intraluminal profile of the HIV cell-free surrogate was similar to that of cell-free HIV surrogate without douching, with the majority of HIV surrogate observed only within the rectosigmoid. These findings support TFV douching as PrEP prior to RAI in further clinical development. This recommendation is also supported by the superior effectiveness of pre-sex PrEP dosing in pre-clinical studies when compared to post-exposure use.

In one participant, when sRAI preceded the TFV douche, the HIV surrogate reached the splenic flexure with a Dₘₐₓ higher than the TFV douche Dₘₐₓ. This suggests the potential for colon lumen redistribution of HIV beyond the distribution of a medicated douche when it follows RAI. Although the rectum may be relatively more susceptible to HIV infection than the colon due to higher expression levels of CCR5, an HIV co-receptor, more proximal redistribution of HIV resulting from RAI before a medicated douche may represent an extended area (to the descending colon) at risk of HIV acquisition. In addition, the colonic mucosal surface can become vulnerable in inflammatory conditions, e.g., Crohn's disease, which may compromise the local epithelial barrier against viral infection. The effector memory subset of CD4+ cells, the largest HIV reservoir in CD4+ cells, is reported to be substantially expanded in individuals with Crohn's disease. Apart from rectal PrEP, a greater concern would be the not uncommon practice of post-RAI douching for cleansing, reported to occur in 27% of survey respondents, which would also be expected to extend the distribution of HIV proximally. Collectively, post-RAI douching, with or without microbicides, may extend HIV surrogate distribution into the descending colon, which may then increase the susceptibility of HIV acquisition via RAI.

Redistribution of colonic luminal HIV with a post-sex douche, however, is only one variable potentially affecting HIV acquisition risk. HIV deposition in the rectosigmoid after unprotected RAI will also be largely diluted by post-RAI non-medicated douching. For example, the sRAI model deposits 2.5 mL of autologous seminal plasma with radiolabeled cell-free HIV surrogate whereas the douche is 125 mL—a 50-fold dilution with idealized, uniform mixing of the two fluids. The tube-fitting analysis estimated that the Cₘₐₓ values of ^{99m} Tc-SC upon post-sRAI douching were 21.8±5.2% of those seen with pre-sRAI douching (Table 3).

Unfortunately, the ^{99m} Tc-SC signal Cₘₐₓ could not reliably provide quantitative estimates of HIV dilution given the complication of SPECT/CT imaging sensitivity and labeling affinity. Understanding the net effects of post-RAI douching on HIV acquisition risk requires further investigation.

Colonic TFV douche distribution without sRAI was reported in DREAM-01. Ranges of TFV douche D_{max, DCmax}, and Dₐᵥₑ from this study are similar to those from DREAM-01, but Dₘᵢₙ values are higher (2.9 to 8.4 cm above the ARJ vs. 0.5 to 6.0 cm above the ARJ in DREAM-01). The earliest SPECT/CT imaging time point in DREAM-01 was at two hours post-dose, while in this study, all SPECT/CT images were acquired at one-hour post-dose. Therefore, douching liquid might gradually descend over time due to bowel movements, resulting in lower Dₘᵢₙ values at later time points. Of note, in DREAM-01, ^{99m}Tc-DTPA was used as the douche surrogate and at a higher radioactive dose than 111 In-DTPA, which might be associated with stronger intensity that were able to capture distal douche surrogate signals. Tube-fitting analysis of SPECT/CT images has been useful in exploring luminal distribution of rectal microbicides. However, this method is subject to numerous sources of inter- and intra-individual variability associated with imaging sensitivity, isotope and carrier dose administered and retained, luminal redistribution/dilution caused by mixing of administered radiolabeled products, and variation in complex colon motility subject to numerous physiologic, and dietary stimuli. In addition, with the small sample size in this study, parameter estimates were not robust. Nevertheless, the study was able to capture an important common trend that seminal fluid distribution would be altered, and most likely, increased upon post-RAI douching.

Since the primary endpoint was intraluminal colonic distribution, plasma TFV was only minimally assessed, opportunistically using plasma collected for radioisotope quantitation during the first hour. A peak in plasma TFV was observed at the first evaluated time point of 20 minutes in all participants, followed by a decline in TFV concentrations throughout the remainder of the first hour; the 0.70 hour half-life is consistent with the estimated plasma TFV distribution half-life (0.71 hrs) from a reported TFV population PK model. This distributional concentration decay is far faster than previously reported plasma TFV elimination half-life (13 to 23 hours). In DREAM-01 (Example 1), which studied the same hypo-osmolar TFV douche without sRAI, the first available plasma TFV concentration was one hour after dosing; these concentrations (interquartile range 4 to 13 ng/mL) largely overlapped the observed one-hour plasma TFV concentration interquartile range in DREAM-02. Beyond the first hour, the observed plasma TFV concentration in DREAM-01 peaked at 3 hours in the hypo-osmolar douche as well as the other two iso-osmolar formulations included in that study. Together, data from DREAM-01 and DREAM-02 suggest a biphasic peak for the hypo-osmolar TFV douche.

The rapid absorption of TFV into plasma, reported here in DREAM-02, may be attributable to the advective forces of the hypo-osmolar fluid moving solute molecules rapidly to the colorectal mucosal surface in the first moments after dosing prior to establishing physiological iso-osmolar concentrations in the colorectal lumen. Following this, a second peak, the one reported in DREAM-01, may be consistent with a slower and sustained absorption from residual drug in the colorectal lumen. Such a biphasic absorption pattern has also been reported with plasma ^{99m}Tc-DTPA in DREAM-01 and is most clearly seen with the hypo-osmolar TFV douche, with a higher initial peak at one hour and a secondary peak at four hours; only the later, second peak was seen with the two iso-osmolar products.

Understanding the time course of this biphasic absorption of rectally administered TFV is crucial to guide dosing regimens for event-driven use of a TFV douche, especially in the complex environment of pre- and post-sex douching associated with RAI. Studies of the impact of douching and sex on the relative distribution and retention of HIV surrogates and other rectal PrEP candidate formulations are needed. The early plasma TFV peak observed is faster than those reported with oral TFV products: oral TDF tablet (TFV Tₘₐₓ 1-3 hours) and oral TAF tablet (TAF Tₘₐₓ 1 hour, TFV Tₘₐₓ 3 hours). The rapid absorption profile of this product positions it as a suitable candidate to meet the on-demand needs of PrEP users. Also, in contrast to oral TFV prodrug dosing, peak TFV concentrations (range: 7 to 22 ng/mL) were observed lower than the trough plasma TFV concentration of oral TDF (range: 27 to 87 ng/ml) and overlapping with the trough plasma TFV concentrations associated with oral TAF (95% CI: 6.9 to 8.6 ng/mL).

Finally, study products and procedure were well tolerated, with only one grade 1 adverse event, which resolved the same day, out of 12 completed study sequences. Previously, DREAM-01, which assessed three different TFV-containing rectal douches, including the one studied in DREAM-02, reported only two study-related grade 1 adverse events out of 60 doses administered. Chronic dosing safety is evaluated in a study which directly compares on demand rectal TFV douching to on demand oral F/TDF dosing.

Compared to administering rectal TFV douche prior to sRAI, post-sRAI use increases the proximal distribution of HIV surrogate from the rectosigmoid into the descending colon with implications for both medicated and non-medicated post-RAI douching as discussed. The hypo-osmolar TFV douche formulation that was tested demonstrates rapid absorption of rectally-applied TFV achieving peak plasma concentrations at least as early as 20 minutes after dosing enabling early phosphorylation and rapid onset of action. These findings suggest preferred TFV douche practices for future clinical development.

In light of the tissue PK and colonic luminal distribution data (DREAM-01, DREAM-02, and DREAM-03), and to allow for as behaviorally congruent use of the TFV douche as possible, it was proposed to allow post-RAI douching during the study with the requirement that it is with a TFV douche. This should maximize the duration of colonic tissue cell TFV-DP, based on findings in DREAM-03.

### Example 4: End-User Product Feasibility DREAM-04

This study of 21 men who douche prior to sex evaluated the feasibility of the anticipated typical TFV douche user to prepare unit doses of the douche from a sachet (pourable powder in single unit dose tear-able foil pouch) formulation.

### Methods

DREAM Program developers selected the sachet as the formulation to test to maximize portability of these discrete, pocket size doses and versatility with douching devices, principally enema bottles and syringe bulbs. In the above cited Grindr behavioral survey, investigators report varying use of enema bottle, syringe bulb, and hose attached to a water source as the most common douching methods. Each method had a different frequency of use in the US, Latin America, and Africa. DREAM-04 was a non-invasive, minimal risk feasibility study which compared end-user performance in preparation of three unit doses of each of two different sachet formulations (lyophilized and spray dried powders) to historic performance of compounding pharmacists in preparation of the douches in DREAM-01 and DREAM-03.

### Results

Overall, 86% of users indicated the lyophilized formulation was either “Very easy” or “Easy” to prepare, while 66% of respondents indicated the spray dried formulation was either “Very easy” or “Easy” to prepare. Of the two products 67% preferred the lyophilized powder product, with 33% reporting no preference. No respondents preferred the spray dried product formulation. In a preliminary analysis of research participant performance making the TFV douche using the lyophilized powder in the foil sachet, 100% of participant-prepared doses met the criteria for color and pH, 89% met the osmolality criteria (145±22 mOsm/kg), and 86% met the TFV concentration criteria (90-110% of 5.28 mg/mL). Performance with the spray-dried formulation was far worse, and attributed largely to pourability challenges with the hygroscopic powder.

While the results of this study demonstrated the feasibility of preparing a TFV douche by the target population, direct comparison of the results of the lyophilized and spray dried formulations was limited by less than optimal features of the spray dried formulation. Specifically, characteristics of the spray dried formulation such as pourability was reduced due to failure to control humidity and clumping of the powder resulting in non-uniform distribution of the active pharmaceutical ingredient within the douching bottle. Similarly, responses to ease-of-use survey questions were most impacted by lack of a easy tear-open pouch design. These were key variables that were not successfully controlled with the resources of an academic pharmaceutics lab. However, the results of this study allowed the team to identify key variables for optimization in a scale-up process that is underway with a Contract Manufacturing Organization (CMO). The drug product being produced in sufficient quantities to support this phase 2 study will have an easy open pouch (perforated for easy, controlled tearing open on one end) and of a pourable powder that can be produced in a commercial facility.

Modification and modifications will be apparent to those skilled in the art and are intended to come within the scope of the appended claims.

We claim:

1. A single dosage form comprising tenofovir or a derivative thereof, in a prophylactically effective amount to inhibit HIV infection within a sex passage of a subject for a period of at least 96 hours following administration into the sex passage.

2. The single dosage form of claim 1, wherein the tenofovir is 9-[9 (R)-2-(phosphonomethoxy) propyl] adenine (PMPA).

3. The single dosage form of claim 1, wherein the tenofovir derivative is selected from the group consisting of tenofovir disoproxil fumarate (TDF), tenofovir exalidex, and tenofovir alafenamide (TAF).

4. The single dosage form of claim 1, wherein the tenofovir or derivative thereof is in an amount of between about 1,000 mg and about 1,500 mg, inclusive, or
wherein the tenofovir or derivative thereof is in an amount of about 1,320 mg.

5. The single dosage form of claim 1, further comprising one or more of NaCl, NaOH, or NaHCO₃,
optionally wherein the final concentration of sodium is between about 0.4% and about 0.5%, inclusive.

6. The single dosage form of claim 1, wherein the tenofovir or derivative thereof is in the form of a solid selected from the group consisting of granules, a powder, a tablet, and lyophilized or dried material.

7. The single dosage form of claim 6, wherein the solid comprises a powder, optionally wherein the powder comprises particles of between about 20 μm to about 600 μm in diameter, inclusive.

8. The single dosage form of claim 7, comprising an amount of a solid form of tenofovir or a derivative thereof, and an amount of sodium chloride to yield, when reconstituted as an aqueous solution, a hypotonic solution of from 145 mOsm/kg to about 290 mOsm/kg, comprising from 0.1 mg/ml to 20 mg/ml, 1.8 mg/ml to 10 mg/ml, 2 mg/ml to 10 mg/ml, or 5.28 mg/ml, of tenofovir or a therapeutic or molar equivalent amount of a derivative thereof.

9. A composition comprising
(i) the single dosage form of claim 1; and
(ii) one or more hermetically sealed container(s), wherein the one or more container comprises a cylindrical profile,
optionally wherein the one or more container comprises a polyester,
optionally wherein the polyester is a biaxially oriented polyethylene terephthalate,
optionally wherein the biaxially-oriented polyethylene terephthalate is metallized.

10. The composition of claim 9, wherein the single dosage form comprises a sterile, lyophilized powder in an amount of about 1,320 mg,
wherein the powder is packaged within the one or more hermetically sealed container,
optionally wherein the one or more hermetically sealed container comprises two compartments, and
wherein each compartment comprises approximately 660 mg of the powder,
optionally wherein the one or more hermetically sealed container comprises a stick pack comprising one or more tear notch at one or more end(s), or
wherein the container one or more hermetically sealed is a sachet comprising one or more tear notch at one or more end(s), and
optionally wherein a tear notch provides an opening to a single compartment of the one or more hermetically sealed container.

11. A method for preparing the single dosage form of tenofovir or a derivative thereof of claim 1, comprising:
(a) dissolving tenofovir or a derivative thereof in water to provide a solution;
(b) adjusting the pH of the solution to a pH ranging from 6.0 to 8.0 or from 6.5 to 7.5;
(c) spray drying the solution under conditions for producing a powder comprising the tenofovir or a derivative thereof; and
(d) mixing the tenofovir or a derivative with a salt or buffer in amounts to produce, when dissolved in water to a volume of between about 240 mL and about 260 mL, inclusive, a prophylactically-effective concentration of the tenofovir or a derivative thereof with an osmolality of about 145±22 milliosmoles per kilogram of water (mOsm/kg H₂O); and
(e) optionally depositing and/or sealing the tenofovir or a derivative within a container.

12. The method of claim 11, wherein the spray drying comprises an inlet temperature of about 200 °C, a pump flow rate of about 4 mL/min, an aspiration rate of about 35 m3/h, and an N2 flow rate of about 601 L/h.

13. A method for administration of the dosage form of claim 1, comprising
administering into the rectum of a subject the effective amount of tenofovir or a derivative thereof to inhibit infection or replication of HIV in the rectum and/or intestine of the subject for about 24 hours to about 96 hours, inclusive, following administration,
wherein the tenofovir or a derivative thereof is administered into the rectum in the form of a hypo-osmotic aqueous solution, and
wherein the hypo-osmotic aqueous solution is applied into the rectum as a first and a second discrete applications, and
wherein the first and second discrete applications are separated by administration of one or more further solutions into the rectum.

14. The method for administration of claim 13, wherein the aqueous solution of the first and/or second discrete applications is retained within the rectum for a period of between about 10 seconds and about 10 minutes, inclusive.

15. The method for administration of claim 14, wherein each of the first and second discrete applications into the rectum comprises administering approximately 50% of the amount of tenofovir or a derivative thereof,
optionally wherein each of the first and second discrete applications into the rectum comprises between about 100 ml and about 150 ml, inclusive, of the aqueous solution.

16. The method for administration of claim 15, comprising one or more discrete application of a further solution into the rectum after the first and before the second discrete applications,
wherein the further solution is selected from the group consisting of water, an aqueous solution of tenofovir or a derivative thereof, saline, and other aqueous solutions.

17. The method for administration of claim 13, further comprising repeating the administration of the dosage form once or more than once.

18. The method for administration of claim 17, wherein the administration is repeated 4 days after the first administration,
optionally wherein the administration is repeated not more than twice a week.

19. A method for reducing or preventing HIV infection associated with receptive sexual intercourse (RSI) in a subject, comprising
(i) administering into the sex passage of the subject from about 20 minutes prior to RSI to about 96 hours prior to RSI, inclusive, a first solution,
wherein the first solution comprises between about 600 mg and about 700 mg inclusive of tenofovir, or a derivative thereof, in a volume of between about 100 ml and about 150 ml, inclusive;
(ii) optionally retaining the first solution within the sex passage for a period of between about 10 seconds and about 10 minutes, inclusive,
(iii) removing/defecating the first solution from the sex passage; and
(iv) administering into the sex passage of the subject from about 20 minutes following RSI to about 60 minutes following RSI, inclusive, a second solution,
wherein the second solution comprises between about 600 mg and about 700 mg inclusive of tenofovir, or a derivative thereof, in a volume of between about 100 ml and about 150 ml, inclusive.

20. The method of claim 19, wherein one or more further solution(s) in an amount between about 1 ml and about 500 ml, inclusive, is administered into the sex passage of the subject following RSI and prior to administering the second solution in (iv), optionally wherein the one or more further solution(s) does not comprise tenofovir or a derivative thereof.

21. The method of claim 19, further comprising
(v) Repeating (i)-(iv) after a period of 24 to 96 hours.

22. The method of claim 21, wherein (i)-(iv) is repeated no more than once in a week.

23. The method of claim 19, wherein the sex passage is a rectum or a vagina.

24. A kit comprising the single dosage form of claim 1, and one or more of
(i) instructions for use;
(ii) a container for administration as an enema; and
(iii) an aqueous solution.
